(19) United States
(12) Patent Application Publication
(10) Pub. No.: US 2025/0043338 A1
(43) Pub. Date: Feb. 6, 2025

(54) PROBE-BASED DEVICE-FREE SINGLE-CELL RNA PROFILING

(71) Applicants: CZ Biohub SF, LLC, San Francisco, CA (US); The Regents of the University of California, Oakland, CA (US)

(72) Inventors: Daniel Austin Foyt, San Francisco, CA (US); Bo Huang, San Francisco, CA (US)

(21) Appl. No.: 18/787,091

(22) Filed: Jul. 29, 2024

Related U.S. Application Data
(60) Provisional application No. 63/517,232, filed on Aug. 2, 2023.

(51) Int. Cl. C12Q 1/6841

(52) U.S. Cl. CPC C12Q 1/6841

(57) ABSTRACT

Provided are methods and compositions for in situ detection of RNA in cells that does not require either the use of reverse transcriptase nor use of droplets. The methods can comprise annealing, in fixed and permeabilized cells, a pair of polynucleotide probes to adjacent sequences in a target RNA, which are subsequently ligated. A cell-specific barcode sequence can be subsequently synthesized in the cell using split-pool rounds to add barcode sequences to the ligated probe pair sequences in the cells, wherein an effect of multiple rounds of the split pooling is that ligated probe pair sequences in different cells have unique barcodes that are cell-specific.

### CROSS-REFERENCES TO RELATED APPLICATIONS

This application claims benefit of U.S. Provisional Patent Application No. 63/517,232 filed Aug. 2, 2023, which application is incorporated herein by reference in its entirety.

### STATEMENT AS TO RIGHTS TO INVENTIONS MADE UNDER FEDERALLY SPONSORED RESEARCH AND DEVELOPMENT

This invention was made with Government support under grant U01 DK127421 awarded by the National Institutes of Health. The Government has certain rights in the invention.

### BACKGROUND OF THE INVENTION

With its ability to profile individual transcriptomes of many cells, single cell RNA sequencing (scRNAseq) has proven to be an invaluable tool in understanding cell to cell heterogeneity and gene regulatory networks in complex systems (1). Most scRNAseq methods capture polyadenylated RNA and then use reverse transcription to convert it into double stranded DNA that is compatible with sequencing reactions (2). Although this approach can analyze mRNAs in an unbiased way, the typical detection efficiencies for individual RNA transcripts ranges between 5-45% (3, 4, 5), largely caused by the inefficiency of the template switching reaction during reverse transcription. These inefficiencies are particularly deleterious for detection of low copy number RNA and lead to drop out or noisy measurements making classification of subtle phenotypes difficult with few cells. (6).

In contrast to the low detection efficiency in scRNAseq, single-molecule fluorescence in situ hybridization (smFISH) regularly achieves a detection efficiency close to 100% by utilizing multiple probes to probe the target RNA directly (7). Taking this concept, single-cell RNA profiling can also be achieved by sequencing multiple in situ hybridization probes for one given transcript to decrease the likelihood of a molecule going undetected and increase the measurement confidence. Indeed, several probe-based single-cell RNA profiling methods have been developed recently, such as HyPR-Seq (8), ProBac-seq (9), and 10× Genomics Chromium Flex protocol (10). Due to their probe-based nature, these methods are inherently targeted, allowing for efficient utilization of sequencing reads, and they are not limited to profiling poly adenylated RNA like many scRNAseq methods. On the other hand, they each have their unique limitations. For instance, their probe chemistry either requires complex oligo hybridization and ligation steps, leading to low probe detection efficiency and high background, or simply relies only on hybridization-based specificity, leading to low specificity. Additionally, all of them use microfluidic partitioning of single cells, which can limit the number of cells profiled and requires costly instrumentation. In contrast, highly scalable methods such as SPLIT-Seq (11) and Sci-Plex (12) can sequence millions of cells by utilizing combinatorial indexing.

### BRIEF SUMMARY OF THE INVENTION

In some embodiments, methods of in situ detection of RNA in cells are provided. In some embodiments, the method comprises,
providing fixed and permeabilized cells in a bulk solution;
in the bulk solution, diffusing single-stranded (ss) DNA probe pairs into the cells and annealing the ssDNA probe pairs to RNA in the cells, wherein the probe pairs comprise a 5′ binding probe and a 3′ binding probe, wherein the 5′ binding probe and the 3′ binding probe anneal to adjacent sequences in a target RNA and wherein,
the 5′ binding probe comprises a 5′ universal sequence that does not anneal to the target RNA and a 3′ RNA annealing sequence and,
the 3′ binding probe comprises a 5′ phosphorylation, a 5′ RNA annealing sequence, and a 3′ adapter sequence that does not anneal to the target RNA;
washing unbound ssDNA probes from the cells;
ligating in the cells annealed probe pairs such that adjacent annealed 5′ binding probes and annealed 3′ binding probes are ligated to form one long probe;
performing a plurality of split-pooling rounds, wherein each round comprises:
(a) aliquoting the cells into a plurality of vessels,
(b) in the vessels, hybridizing a double-stranded (ds) barcoding oligonucleotide comprising (i) a first overhang sequence and (ii) a central double-stranded sequence having a barcode sequence and (iii) a second overhang sequence, wherein the first overhang anneals to a 3′ or 5′ end of the long probe and,
(c) in the vessels, ligating a strand of the ds barcoding oligonucleotide to the 3′ or 5′ end of the long probe to form barcoded ligated products, and
(d) combining the contents of the vessels to form a bulk solution comprising cells containing the ligated products,
wherein the plurality of split-pooling rounds forms cell-specific barcoded long probe polynucleotides; and
nucleotide sequencing the cell-specific barcoded long probe polynucleotides.

In some embodiments, the hybridizing comprises hybridizing a double-stranded (ds) barcoding oligonucleotide comprising (i) a 3′ first overhang sequence and (ii) a central double-stranded sequence having a barcode sequence and (iii) a second 3′ overhang sequence, wherein the 3′ first overhang anneals to a 3′ end of the long probe; and the ligating comprises ligating a strand of the ds barcoding oligonucleotide to the 3′ end of the long probe to form barcoded ligated products.

In some embodiments, the hybridizing comprises hybridizing a double-stranded (ds) barcoding oligonucleotide comprising (i) a 5′ first overhang sequence and (ii) a central double-stranded sequence having a barcode sequence and (iii) a second 5′ overhang sequence, wherein the 5′ first overhang anneals to a 5′ end of the long probe; and the ligating comprises ligating a strand of the ds barcoding oligonucleotide to the 5′ end of the long probe to form barcoded ligated products.

In some embodiments, a first round of split-pooling comprises:
aliquoting cells from the bulk solution into a plurality of vessels;
in the vessels annealing a first double-stranded (ds) barcoding oligonucleotide comprising (i) a 3′ overhang sequence that anneals to the 3′ adapter sequence on the long probe and (ii) a central double-stranded sequence having a first barcode sequence and (iii) a 3′ overhang sequence comprising a first linking sequence,
in the vessels, ligating the first ds barcoding oligonucleotide to the 3′ adapter sequence on the long probe to form a first partially barcoded long probe comprising the first barcode sequence and a 3′ end having the first linking sequence;
combining the contents of the vessels to form a second bulk solution.

In some embodiments, the method further comprises a second round of split-pooling after the first round, the second round comprising,
aliquoting cells from the second bulk solution into a new plurality of vessels;
in the vessels annealing a second double-stranded (ds) barcoding oligonucleotide comprising (i) a 3′ overhang sequence that anneals to the first linking sequence and (ii) a central double-stranded sequence having a second barcode sequence and (iii) a 3′ overhang sequence comprising a second linking sequence;
in the vessels, ligating the second ds barcoding oligonucleotide to the first linking sequence on the long probe to form a second partially long probe comprising the first and second barcode sequence and a 3′ end having the second linking sequence; and
combining the contents of the vessels to form a third bulk solution.

In some embodiments, the method further comprises a third round of split-pooling after the second round, the third round comprising,
aliquoting cells from the third bulk solution into a new plurality of vessels;
in the vessels annealing a third double-stranded (ds) barcoding oligonucleotide comprising (i) a 3′ overhang sequence that anneals to the second linking sequence and (ii) a central double-stranded sequence having a third barcode sequence and (iii) a 3′ overhang sequence comprising a third linking sequence;
in the vessels, ligating the third ds barcoding oligonucleotide to the second linking sequence on the long probe to form a third long probe comprising the first and second and third barcode sequence and a 3′ end having the third linking sequence; and
combining the contents of the vessels to form a fourth bulk solution.

In some embodiments, the method further comprises, before the nucleotide sequencing, amplifying the cell-specific barcoded long probe polynucleotides with (i) a first primer that anneals to the 5′ universal sequence or a complement thereof and (ii) a second primer that anneals to a 3′ sequence of the cell-specific barcoded long probe polynucleotides or a complement thereof to form an amplicon.

In some embodiments, a first round of split-pooling comprises:
aliquoting cells from the bulk solution into a plurality of vessels;
in the vessels annealing a first double-stranded (ds) barcoding oligonucleotide comprising (i) a 5′ overhang sequence that anneals to the 5′ universal sequence on the long probe and (ii) a central double-stranded sequence having a first barcode sequence and (iii) a 5′ overhang sequence comprising a first linking sequence,
in the vessels, ligating the first ds barcoding oligonucleotide to the 5′ universal sequence on the long probe to form a first partially barcoded long probe comprising the first barcode sequence and a 5′ end having the first linking sequence;
combining the contents of the vessels to form a second bulk solution.

In some embodiments, the method further comprises a second round of split-pooling after the first round, the second round comprising,
aliquoting cells from the second bulk solution into a new plurality of vessels;
in the vessels annealing a second double-stranded (ds) barcoding oligonucleotide comprising (i) a 5′ overhang sequence that anneals to the first linking sequence and (ii) a central double-stranded sequence having a second barcode sequence and (iii) a 5′ overhang sequence comprising a second linking sequence;
in the vessels, ligating the second ds barcoding oligonucleotide to the first linking sequence on the long probe to form a second partially long probe comprising the first and second barcode sequence and a 5′ end having the second linking sequence; and
combining the contents of the vessels to form a third bulk solution.

In some embodiments, the method further comprises a third round of split-pooling after the second round, the third round comprising,
aliquoting cells from the third bulk solution into a new plurality of vessels;
in the vessels annealing a third double-stranded (ds) barcoding oligonucleotide comprising (i) a 5′ overhang sequence that anneals to the second linking sequence and (ii) a central double-stranded sequence having a third barcode sequence and (iii) a 5′ overhang sequence comprising a third linking sequence;
in the vessels, ligating the third ds barcoding oligonucleotide to the second linking sequence on the long probe to form a third long probe comprising the first and second and third barcode sequence and a 5′ end having the third linking sequence; and
combining the contents of the vessels to form a fourth bulk solution.

In some embodiments, the method further comprises, before the nucleotide sequencing, amplifying the cell-specific barcoded long probe polynucleotides with (i) a first primer that anneals to the 3′ adapter sequence or a complement thereof and (ii) a second primer that anneals to a 5′ sequence of the cell-specific barcoded long probe polynucleotides or a complement thereof to form an amplicon.

In some embodiments, the amplifying occurs in a plurality of vessels. In some embodiments, the second primers comprise a further vessel-specific barcoding sequence. In some embodiments, the first primer and the second primers comprise 5′ sequences that introduce sequencing adapter sequences to the amplicon.

In some embodiments, at least 2, 3, 4, 5, or more (e.g., 2-6, 2-10, 2-20, 6-20, 10-50) different ss DNA probe pairs are targeted to different sequences on the same target RNA.

In some embodiments, different ss DNA probe pairs are targeted to different target RNAs. In some embodiments, different target RNAs have different expression levels and more ss DNA probe pairs are targeted to lower-expressing target RNAs compared to ss DNA probe pairs are targeted to higher-expressing target RNAs.

In some embodiments, a PBCV-1 DNA ligase catalyzes the ligating of the annealed probe pairs in the cells.

In some embodiments, each barcode sequence in the ds barcoding oligonucleotide is between 4-10 nucleotides each.

In some embodiments, the sum of the lengths of the 3′ RNA annealing sequence and the 5′ RNA annealing sequence is 20-40 nucleotides long. In some embodiments, the 3′ RNA annealing sequence and the 5′ RNA annealing sequence are each 14-16 nucleotides long.

In some embodiments, the first and second nucleotide of the 5′ RNA annealing sequence is A or T.

In some embodiments, the % GC of the 3′ RNA annealing sequence and the 5′ RNA annealing sequence is 30-70%.

In some embodiments, the ligating of the strand of the ds barcoding oligonucleotide to the 3′ end of the long probe is catalyzed by a T4 ligase.

In some embodiments, the vessels are in a microtiter multi-well plate.

Also provided are reaction mixtures for use in the methods as described above and elsewhere herein. In some embodiments, the reaction mixture comprises fixed and permeabilized cells and single-stranded (ss) DNA probe pairs diffused into the cells, wherein at least some of the ss DNA probe pairs anneal to RNA in the cells, wherein the probe pairs comprise a 5′ binding probe and a 3′ binding probe, wherein the 5′ binding probe and the 3′ binding probe anneal to adjacent sequences in a target RNA and wherein, the 5′ binding probe comprises a 5′ universal sequence that does not anneal to the target RNA and a 3′ RNA annealing sequence and, the 3′ binding probe comprises a 5′ phosphorylation, a 5′ RNA annealing sequence, and a 3′ adapter sequence that does not anneal to the target RNA.

Also provided are kits for use in the methods as described above and elsewhere herein. In some embodiments, the kit comprises at least 100 different single-stranded (ss) DNA probe pairs that anneal to different RNA from a cell, wherein the probe pairs comprise a 5′ binding probe and a 3′ binding probe, wherein the 5′ binding probe and the 3′ binding probe anneal to adjacent sequences in a target RNA and wherein, the 5′ binding probe comprises a 5′ universal sequence that does not anneal to the target RNA and a 3′ RNA annealing sequence and, the 3′ binding probe comprises a 5′ phosphorylation, a 5′ RNA annealing sequence, and a 3′ adapter sequence that does not anneal to the target RNA; and a plurality of double-stranded (ds) barcoding oligonucleotides comprising (i) a first overhang sequence and (ii) a central double-stranded sequence having a barcode sequence and (iii) a second overhang sequence.

### BRIEF DESCRIPTION OF THE DRAWINGS

FIG. 1A-D is a schematic diagram of some of the methods descried herein. 1A) Multiple split probes are designed per transcript of interest. 1B) Hybridization and ligation of split probes. 1C) Labeling probes with unique cell barcode via split and pool method. Rounds 1 and 2 barcodes are ligated, and round 3 barcodes are added via PCR. 1D) Sequence structure for the resulted library.

FIG. 1E depicts HybriSeq sequencing results of 1:1 mixed HEK293 cells stably expressing either mNG or GFP.

FIG. 1F depicts a scatter plot of cell line matched bulk RNA-Seq TMP number and HybriSeq average UMIs/cell for 95 cell cycle associated genes. Each point represents a transcript measured. r (Pearson correlation coefficient).

FIG. 1G depicts a scatter plot of average HybriSeq UMIs/HEK293 cell in two independent biological replicates. Each dot represents a single probe for either GAPDH, RPL13A, or ACTB. r (Pearson correlation coefficient).

FIG. 2A depicts schematic of initial probe design and RNAseH specificity method. Our initial approach was to hybridize non-split probes and qPCR/sequences them directly or release RNA bound probes with RNAseH.

FIG. 2B depicts bulk UMIs/nontarget probes (30 nt) for probes targeting transduced fluorescent protein transcripts in HEK293 cells. Cells were directly loaded into a limited cycle PCR to amplify cell associated probes. Probes were sequenced. Each point represents a unique probe.

FIG. 2C depicts a scatter plot of bulk scaled expression values and RNA-Seq scaled expression values for genes measured with non split probes (30 nt) released via RNAseH. ˜20 probes were used to detect the same transcript and there UMIs were averaged together to yield an expression value. Each point represents a gene measured colored by average expression above nontargeting probes. Pearson correlation coefficient r=0.767.

FIG. 2D depicts hybridization condition optimization for non split probes released via RNAseH of lengths 30 nt and 45 nt. Concentrations of RNAaseH released probes targeting FLNA RNA or nontargeted in HELA cells measured with qPCR. Error bars represent the standard deviation associated with technical replicates. % formamide is the concentration of formamide used in the hybridization buffer and temperature (X axis) represent the temperature used during hybridization.

FIG. 2E depicts concentration of RNAaseH released probes (30 nt) targeting FLNA RNA or nontargeted in HELA cells measured with qPCR for 1-1000 nM hybridization concentration.

FIG. 3A depicts a schematic of split probe design and ligation specificity method.

FIG. 3B depicts bulk concentration of ligated probes targeting TUBB4B RNA or nontargeted in HELA cells measured with qPCR for 0.1-1000 nM split probe hybridization concentration. For hybridization concentration 0.1-100 nM the nontargeting probes were below the limit of detection. Error bars represent the standard deviation associated with technical replicates.

FIG. 3C depicts bulk concentration of ligated and left (unligated) probes targeting TUBB4B RNA in HELA cells measured with qPCR for 0.1-1000 nM split probe hybridization concentration. Error bars represent the standard deviation associated with technical replicates.

FIG. 3D depicts bulk fraction of misligation events/# of probes in library. Misligation is the result of a left and right probe ligating that do not target adjacent region of a transcript. Error bars represent the standard deviation associated with biological replicates n=3.

FIG. 3E depicts Pearson correlation coefficient (r) for correlation between bulk HybriSeq counts and RNA-Seq expression values for matched cell lines. Concentration refers to SplintR ligase used to ligate probes. Error bars represent the standard deviation associated with biological replicates n=3.

FIG. 4A depicts initial percentage of probe ligated ends for original round 1 barcode ligation conditions (15 min, 25 C, 200 nM CBC & linker) measured in bulk with qPCR. Error bars represent the standard deviation associated with biological replicates n=3.

FIG. 4B depicts percentage of probe ligated ends for round 1 barcode ligation for ligation times 15-240 minutes (min) and barcode/linker concentration in reactions of 50 nM, 100 nM, 200 nM, and 400 nM. Measured in bulk with qPCR.

FIG. 4C depicts a schematic of initial blocking strategy. Blocking oligos are used to displace linker oligos and prevent them from participating in ligation during subsequent rounds of cell barcoding.

FIG. 4D depicts a schematic of HybriSeq quenching strategy. Quench oligos are used to ligate onto the ends of free unligated barcodes thereby preventing them from participating in subsequent ligation reactions.

FIG. 4E depicts Barcode oligos/linker were either blocked or quenched, cells with probes with free unligated 3′ ends were added and allowed to react with blocked or quenched barcodes for 2 h and the fraction of ligated and unligated probe 3′ ends were measured with qPCR. Error bars represent the standard deviation associated with technical replicates.

FIG. 4F depicts cells went through 2 rounds of ligation based barcoding with one of two barcodes in each round. In each pooling step the unreacted or quenched other barcodes was added and allowed to react. Cells were either washed 2 times in EDAT solution after pooling or not washed and put into the next round of barcoding. The quantity of each possible barcode was measured with qPCR and the percent incorrect barcode was calculated for each condition.

### DEFINITIONS

As used herein, the terms “a”, “an”, and “the” can refer to one or more unless specifically noted otherwise.

A “polynucleotide” or “nucleic acid” includes any form of RNA or DNA, including, for example, genomic DNA; complementary DNA (cDNA); DNA molecules produced by amplification; or synthetically produced DNA or RNA molecules. The terms include chimeric molecules and molecules comprising non-standard bases, modifications, or nucleotide analogs. For example, an oligonucleotide may contain naturally occurring nucleotides and/or analogs thereof. Polynucleotides may be single-stranded or double-stranded.

As used herein, the term “barcode” or “BC” refers to a short (typically less than 50 bases, often less than 30 bases) nucleic acid sequence that identifies a property of a polynucleotide. For example, in some cases polynucleotides with the same barcode have a common origin, e.g., are from the same vessel or compartment. While reference may be made to a barcode sequence,” it will be appreciate that in the context of s double-stranded nucleic acid there is a barcode sequence and a barcode sequence complement. It will be recognized that in a double-stranded polynucleotide the sequence in both strands is informative and can serve as a barcode.

Barcodes can be delivered as part of a sequence of an oligonucleotide that is subsequently attached to a polynucleotide to be barcoded. The barcode sequences may vary in length, e.g., depending on the number of target polynucleotides. In certain embodiments, the barcode sequences can have a length, for example, of 4, 5, 6, 7, 8, 9, 10, 11, 12, 13, 14, 15 nucleotides, or longer. As described herein, in some embodiments, barcode sequences are added sequentially (e.g., as part of a split-pool approach) and thus, 2, 3, 4, or more barcode sequences are linked sequentially to a target polynucleotide, and the sum of the barcode sequences creates a unique barcode (e.g., a cell-specific barcode). The oligonucleotides may be DNA, RNA, a combination, or may comprise one or more non-naturally occurring nucleotides, nucleotide analogs, or and/or chemical modifications. Non-naturally occurring nucleotides and/or nucleotide analogs can be modified at the ribose, phosphate, and/or base moiety. Examples of modified base moieties include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, 1-methylguanine, 1-methylinosine, 2,2-dimethylguanine, 2-methyladenine, 2-methylguanine, 3-methylcytosine, 5-methyl cytosine, N6-adenine, 7-methylguanine, 5-methylaminomethyluracil, methoxyarninomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acidmethylester, uracil-S-oxyacetic acid, 5-methyl-2-thiouracil, 3-(3-amino-3-N-2-carboxypropyl) uracil, 2,6-diaminopurine and biotinylated analogs, amongst others. Examples of modified sugar moieties include, but are not limited to, arabinose, 2-fluoroarabinose, xylose, and hexose, or a modified component of the phosphate backbone, such as a phosphorothioate, a phosphorodithioate, a phosphoramidothioate, a phosphoramidate, a phosphordiamidate, a methylphosphonate, an alkylphosphotriester, or a formacetal or analog thereof. In some embodiments, an oligonucleotide can comprise one or more ribonucleotides and one or more deoxyribonucleotides. In some embodiments the oligonucleotide may comprise a boranophosphate linkage, a locked nucleic acid (LNA) nucleotide, a peptide nucleic acid (PNA), or bridged nucleic acids (BNA). The oligonucleotide may comprise regions in addition to the barcode sequence that include, but are not limited to, primer binding sites for sequencing primers, primer binding sites for subsequent amplification, and a unique molecular identifier sequence (UMI) specific for the molecule or as otherwise described herein.

As used herein, the term “vessel” refers to a container in which a solution containing cells, oligonucleotides, and/or constructs can be pooled (combined). Antibody binding and nucleic acid hybridization may occur in a vessel. The term “vessel” does not imply a particular structure or material. Examples of vessels include tubes, wells, microwells, and microfluidic chambers.

### DETAILED DESCRIPTION OF THE INVENTION

The inventors have developed a new method of in situ detection of RNA in cells that does not require either the use of reverse transcriptase nor use of droplets. The method comprises annealing, in fixed and permeabilized cells, a pair of polynucleotide probes to adjacent sequences in a target RNA, which are subsequently ligated. A cell-specific barcode sequence can be subsequently synthesized in the cell using split-pool rounds to add barcode sequences to the ligated probe pair sequences in the cells, wherein an effect of multiple rounds of the split pooling is that ligated probe pair sequences in different cells have unique barcodes that are cell-specific. The resulting polynucleotide product can have sequencing adapter sequences added to either end, for example via amplification with appropriate primers, and be nucleotide sequenced. The identity and quantity of amplified products can then be used to indicate the presence and/or quantity of RNA targets in the cells.

The initial steps of the assay can occur in situ, meaning the assay will measure target RNA as they occur in the cells themselves. The cells can be part of a tissue or can be individual cells. The cells in some embodiments are from primary tissue or are primary cells. In some embodiments, cells are eukaryotic cells, including, but not limited to, yeast and fungi cells, plant cells, avian cells, mammalian cells, and the like. In some embodiments, the cells are mammalian cells, e.g., human cells. In some embodiments, the cells are cancer cells, stem cells, neurological cells, peripheral blood mononuclear cells, lymphocytes, or cells from a cell line. In some embodiments, the cells are obtained from a tissue e.g., a human tissue. In some embodiments, the cells are obtained from a tumor, e.g., a human tumor.

The cells can be fixed and permeabilized by any desired method. The term “fixing” or “fixation” as used herein is the process of preserving biological material (e.g., tissues, cells, organelles, molecules, etc.) from decay and/or degradation. Fixation may be accomplished using any convenient protocol. Fixation can include contacting the cellular sample with a fixation reagent (i.e., a reagent that contains at least one fixative). Cellular samples can be contacted by a fixation reagent for a wide range of times, which can depend on the temperature, the nature of the sample, and on the fixative(s). For example, a cellular sample can be contacted by a fixation reagent for 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes. In some embodiments, a cellular sample can be contacted by a fixation reagent at a temperature ranging from 22° C. to 55° C. Any convenient fixation reagent can be used.

Exemplary fixation reagents include for example crosslinking fixatives, precipitating fixatives, oxidizing fixatives, mercurials, and the like. Crosslinking fixatives chemically join two or more molecules by a covalent bond and a wide range of cross-linking reagents can be used. Examples of suitable cross-liking fixatives include but are not limited to aldehydes (e.g., formaldehyde, also commonly referred to as “paraformaldehyde” and “formalin”; glutaraldehyde; etc.), imidoesters, NHS (N-Hydroxysuccinimide) esters, and the like. Examples of suitable precipitating fixatives include but are not limited to alcohols (e.g., methanol, ethanol, etc.), acetone, acetic acid, etc. In some embodiments, the fixative is formaldehyde (i.e., paraformaldehyde or formalin). A suitable final concentration of formaldehyde in a fixation reagent is 0.1 to 10%, 1-8%, 1-4%, 1-2%, 3-5%, or 3.5-4.5%. In some embodiments the cellular sample is fixed in a final concentration of 4% formaldehyde (as diluted from a more concentrated stock solution, e.g., 38%, 37%, 36%, 20%, 18%, 16%, 14%, 10%, 8%, 6%, etc.). In some embodiments the cellular sample is fixed in a final concentration of 10% formaldehyde. In some embodiments the cellular sample is fixed in a final concentration of 1% formaldehyde. In some embodiments, the fixative is glutaraldehyde. A suitable concentration of glutaraldehyde in a fixation reagent is 0.1 to 1%. A fixation reagent can contain more than one fixative in any combination. For example, in some embodiments the cellular sample is contacted with a fixation reagent containing both formaldehyde and glutaraldehyde.

Cells will in some embodiments also be permeabilized to allow for diffusion of smaller regents in and out of the cells while substantially retaining larger macromolecules in the cell. The terms “permeabilization” or “permeabilize” as used herein refer to the process of rendering the cells (cell membranes etc.) of a cellular sample permeable to experimental reagents such as nucleic acid probes, antibodies, chemical substrates, etc. Any convenient method and/or reagent for permeabilization can be used. Suitable permeabilization reagents include detergents (e.g., Saponin, Triton X-100, Tween-20, etc.), organic fixatives (e.g., acetone, methanol, ethanol, etc.), enzymes, etc. Detergents can be used at a range of concentrations. For example, 0.001%-1% detergent, 0.05%-0.5% detergent, or 0.1%-0.3% detergent can be used for permeabilization (e.g., 0.1% Saponin, 0.2% tween-20, 0.1-0.3% triton X-100, etc.). In some embodiments, the same solution can be used as the fixation reagent and the permeabilization reagent. For example, in some embodiments, the fixation reagent contains 0.1%-10% formaldehyde and 0.001%-1% saponin. In some embodiments, the fixation reagent contains 1% formaldehyde and 0.3% saponin.

In some embodiments, a cellular sample is contacted with an enzymatic permeabilization reagent. Enzymatic permeabilization reagents that permeabilize a cellular sample by partially degrading extracellular matrix or surface proteins that hinder the permeation of the cellular sample by assay reagents. Contact with an enzymatic permeabilization reagent can take place at any point after fixation and prior to target detection. In some instances the enzymatic permeabilization reagent is proteinase K, a commercially available enzyme. In such cases, the cellular sample is contacted with proteinase K prior to contact with a post-fixation reagent (described below). Proteinase K treatment (i.e., contact by proteinase K; also commonly referred to as “proteinase K digestion”) can be performed over a range of times at a range of temperatures, over a range of enzyme concentrations that are empirically. Contact of a cellular sample with at least a fixation reagent and a permeabilization reagent results in the production of a fixed/permeabilized cellular sample.

The fixed and permeabilized cells are provided in a bulk solution, meaning that the cells are in a solution together. For example, the cells are not what one would consider “partitioned.” For example, the cells are not partitioned in droplets or microwells.

Single-stranded (ss) DNA probe pairs are subsequently diffused into the fixed and permeabilized cells at a sufficient concentration to anneal to target sequences of RNA in the cells, if the RNA is present. Any target RNA in the cell can be pargeted as desired. In preferred embodiments, a number of different RNAs in the cell can be targeted, each by a separate ssDNA probe pair. Thus in some embodiments, at least 1, 2, 5, 10, 20, 50, 100, 1000 or more distinct RNAs are targeted, and thus for each RNA at least one ss DNA probe pair is provided.

In some embodiments, one can use different quantities of ss DNA probe pairs for different target sequences in RNA, depending upon the expression level or expected expression levels of the different RNA targets. Specifically, one can use more ss DNA probe pairs to target a low expression RNA while using few ss DNA probe pairs for an RNA target with higher or expected higher expression. The number of unique (in sequence) probes per unique transcript being profiled can be varied based on the expected number of said transcripts in a cell, not the concentration of probes per transcript in the hybridization. For example if we expect a population of cells to have 100 copies/cell of RNA1 and RNA1 has 2 unique (in sequence) probes, we would expect 200 RNA1 probes/cell (100 transcripts×2 probes/transcript) to hybridize to those cells. But if in the same population of cells we expect 10 copies/cell of RAN2, we can include 20 unique probes for RNA2 and we would expect 200 RNA2 probes/cell (10 transcripts×20 probes/transcript) to hybridize to those cells. This can result in mor efficient use of sequencing reads.

To improve sensitivity, one can further target a particular RNAs with different ss DNA probe pairs where different primer pairs target different sequences in the RNA. Thus for example, at least 2, 3, 4, 5, 6, 7, 8, 9, 10 or more (e.g., 2-6, 2-10, 2-20, 6-20, 10-50) different ss DNA probe pairs can target different sequences in the same target RNA. This can improve sensitivity for a particular target RNA. In some embodiments, a larger number of different probes are used to target RNA sequences expected to have relative low abundance compared to different RNA targets for which a lower number of different probes are used.

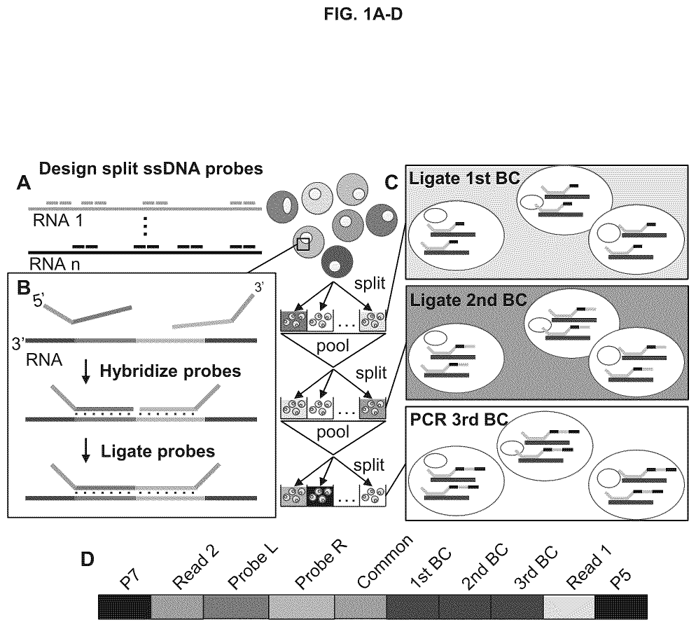

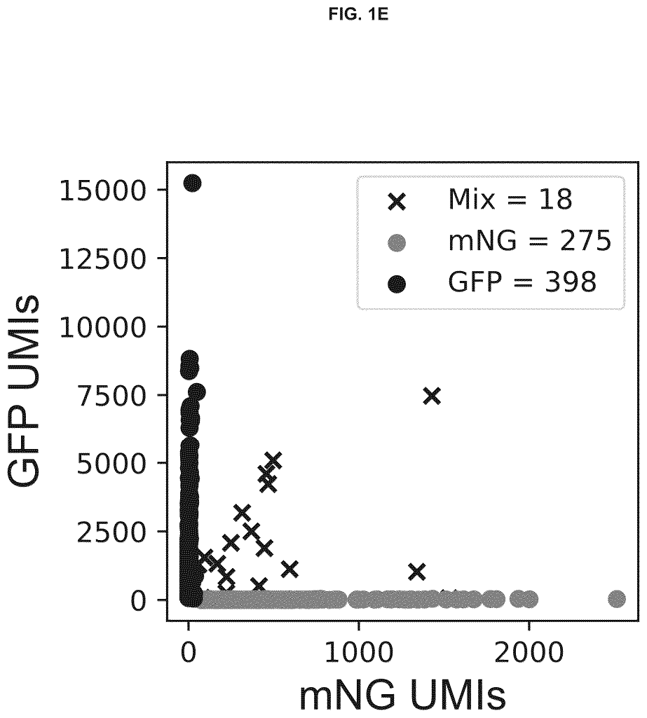

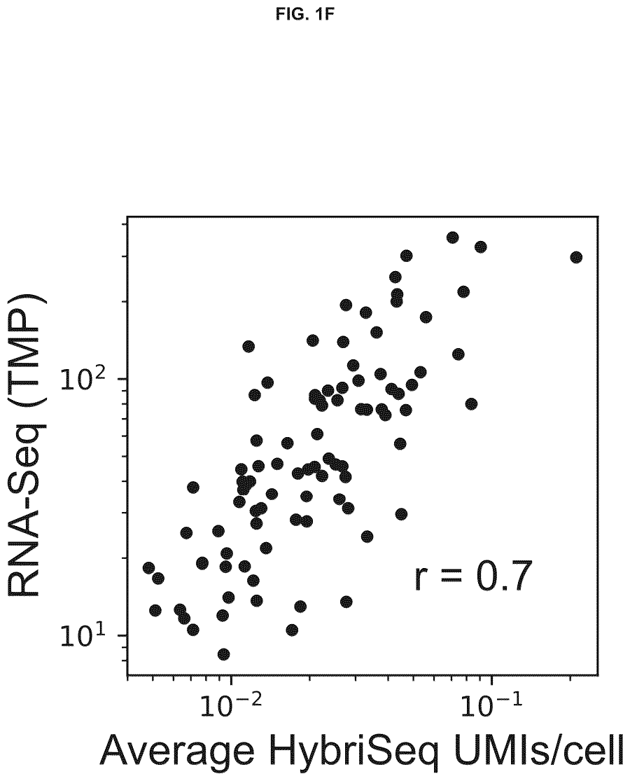

The different ss DNA probe pairs are composed of a 5′ binding probe oligonucleotide and a 3′ binding probe oligonucleotide. The 5′ binding probe comprises a 5′ universal sequence that does not anneal to the target RNA and a 3′ RNA-annealing sequence. The 3′ binding probe comprises a 5′ phosphorylation, a 5′ RNA annealing sequence, and a 3′ adapter sequence that does not anneal to the target RNA. The 3′ binding probe has a 5′ phosphate so that the 5′ end can be ligated to the 3′ end of the 5′ binding probe in a subsequent step. The 3′ RNA-annealing sequence and the 5′ RNA-annealing sequence are designed to anneal to adjacent sequences in a target RNA. “Adjacent” means there are no intervening nucleotides between the two RNA sequences to which the 3′ RNA-annealing sequence and the 5′ RNA-annealing sequence anneal, allowing for subsequent ligation of the annealed DNA sequences as described further below. See, e.g., FIG. 1B. Hybridization conditions can be readily selected for annealing the probes to the RNA, for example such that annealing only significantly occurs if the RNA has a completely complementary sequence. Exemplary hybridization buffer can comprise for example: 30% (v/v) formamide, 1% (w/v) Bovine serum albumin, 0.5% (v/v) Tween 20, 2× Sodium Citrate Buffer, 40 U/ml Rnasin). Exemplary hybridization can occur over 12-24 hours. An exemplary probe concentration is: 10 nM/probe. Because the subsequent ligation only occurs if both probe sequences anneal to adjacent sites on the RNA, yet another level of selectivity for specific binding is included in the method in addition to specific hybridization of the probes. One advantage of the methods described herein is that two adjacent sequences must be annealed to a target and be ligated, greatly reducing non-specific signal that can occur when only single annealed probes are required for detection.

Both the 5′ probe and the 3′ probe comprise sequences that do not anneal to the target RNA and these sequences are used in subsequent steps. The 5′ universal sequence in the 5′ probe is a sequence that will later be used as a universal primer site, allowing for amplification of the product following barcoding as detailed below, or in other embodiments (e.g., where the barcode is added to the 5′ end, the 5′ universal sequence will function as an adapter). The 3′ adapter sequence in the 3′ probe provides a sequence available for hybridization of the first barcoding oligonucleotide in the split-pooling steps as described below when the barcode is added to the 3′ end or when the barcode is added to the 5′ end the 3′ adapter sequence will function like a universal sequence, being available as an amplification primer annealing site for amplifying a population of nucleic acids.

The length of the various sequences of the 5′ and 3′ probes can vary as desired by the user. In some embodiments, the 5′ universal sequence and the 3′ adapter sequences can be between, for example, 8-30 nucleotides. The length of the 5′ universal sequence and the 3′ adapter sequences need not be the same. The length of the 3′ RNA-annealing sequence and the 5′ RNA-annealing sequence is selected such that the combined product has the desired specificity for binding to the target RNA without substantially annealing to non-target RNA. In some embodiments, the sum of the length of the 3′ RNA-annealing sequence and the 5′ RNA-annealing sequence is 20-40 nucleotides long. For example, in some embodiments, the 3′ RNA-annealing sequence and the 5′ RNA-annealing sequence are each 14-16 nucleotides in length though of course other lengths are also possible.

In some embodiments, the first or second or both first and second 5′ end nucleotides of the 5′ RNA-annealing sequence are A or T. This can improve efficiency of the subsequent ligation because certain ligases are more efficient if these positions are A or T (i.e., not G or C).

Once the probes have been annealed to the RNA in the cell, unbound probe can optionally be washed away, before proceeding further, for example by exposing the cells to dilution. Exemplary wash conditions can include, for example, a wash buffer (20%-30% formamide, 0.5% tween 20, 4× Sodium Citrate Buffer, 40 U/ml Rnasin, optionally at 37° C. for about 5 min with gentle agitation, and optionally repeated two or three times.

Following annealing of the probes (and optional wash) the cells can be contacted with a ligase that ligates adjacent annealing polynucleotides annealed to RNA. In some embodiments, the ligase is selected such that the ligase preferable ligates DNA sequences annealed to RNA. Exemplary ligase can include for example, PBCV-1 DNA ligase. See, for example, ligases as described in U.S. Pat. No. 10,597,650. An exemplary PBCV-1 DNA ligase sold commercially as SplintR™ ligase is available from New England Biolabs. Exemplary ligation conditions can last, for example, for 1-4 hours, for example at 25 degrees C. In some embodiments, oligonucleotides are provided at a concentration of for example, 400 nM-1 μM.

Once the 5′ probe and the 3′ probe (annealed to the target RNA) have been ligated, the resulting probe is referred to herein as a “long probe” for convenience. The term “long” in this context does not connote a particular length and instead means the probe is long compared to either of the individual 5′ and 3′ probes.

Following ligation, further washes can be performed, if desired, to remove non-ligated probes. However, this is not necessary as later steps will amplify the barcodes long probe sequences that will only occur for ligation products that are subsequently barcoded as described herein.

Split pooling in the fixed and permeabilized can be used to attach cell-specific barcode sequences to either or both ends of the long probes. This can be achieved for example by aliquoting a solution of the cells into individual wells or other vessels that contain unique barcoding oligonucleotides, linking the barcoding oligonucleotides to the long probes in the cells, then forming a bulk solution from the resulting cells, and repeating the aliquoting and linking process in a sufficient number of times such that each cell contains a unique barcode sequence on long probes the cell contains. Various methods of adding barcodes in nucleic acids in cells have been described, including in but not limited to U.S. Pat. No. 11,634,752 and U.S. Patent publication No. 2022/0403452. The vessels can be for example, wells in a micro-well plate, for example, but not limited to 96-well plates.

FIG. 1C. depicts an exemplary split pool approach. In some embodiments, in each round of split-pooling, a double-stranded (ds) barcoding oligonucleotide is linked to the 3′ end of the long probe or, in second or further split-pooling rounds, the 3′ end of barcoding sequences added to the long probe. For example, in some embodiments, the ds barcoding oligonucleotide having two 3′ overhang sequences such that the ds barcoding oligonucleotide comprises:
(i) a 3′ first overhang sequence that anneals to the 3′ end of the long probe or, in second or further split-pooling rounds, the 3′ end of barcoding sequences added to the long probe, and
(ii) a central double-stranded sequence having a barcode sequence, and
(iii) a second 3′ overhang sequence, which can be used to link a further ds barcoding oligonucleotide in further rounds, with the second 3′ overhang sequence in the last round being available to anneal to primers in the amplification reaction noted below.

While the example above describes an embodiment in which the ds barcoding oligonucleotide is linked to the 3′ end of the long probe, it will be appreciated the same reaction can be performed in which the ds barcoding oligonucleotide has 5′ overhang sequences such that the ds barcoding oligonucleotide is hybridized and ligated to the 5′ end of the long probe. Thus, as desired, barcoding can occur at either end of the long probe. Each round of split-pooling will add a barcode sequence and after sufficient rounds the cumulative barcode sequence (i.e., the product of several linked barcode sequences) will be unique for the cell in which the cumulative barcode resides.

The lengths and composition of the first and second overhang sequences (3′ or 5′ overhangs depending on which end of the long probe is to be annealed to) and the barcode sequences in the ds barcoding oligonucleotide can be selected as desired. In some embodiments, the first and second overhangs each are of 4-20 nucleotides long. The length of the barcode sequence in the ds barcoding oligonucleotide can vary for example depending on the complexity and number of split-pooling rounds and the number of cells involved. In some embodiments, the barcode sequence is 4-20 nucleotides (base pairs) long. Similarly, the overhangs can be for example, 4-20 nucleotides in length. The barcode sequences are selected in such a way that they are different enough to tolerate incorrect base calling during sequencing. Specifically they can be selected to have a Hamming distance >=2 from any other barcode sequence in the barcoding round.

Adding of the barcoding sequences can occur, for example by annealing followed by ligation. In some embodiments, the ligation conditions can comprise 400 nM-1 μM linker/barcode oligonucleotide in ligase buffer (e.g., 1×T4 DNA Ligase Reaction Buffer (NEB), 0.4 Mm ATP, 40 U/ml Rnasin, 0.5% tween 20, 1% BSA, 200,000 U/ml T4 ligase) at 25 C for 1-4 hours. Unlike the annealing of the 5′ and 3′ probes to form the long probe, in which PBCV-1 DNA ligase may be preferred (DNA/RNA hybrids), in ligation of barcode sequences to the long probe (all DNA hybrids), T4 ligase can be used.

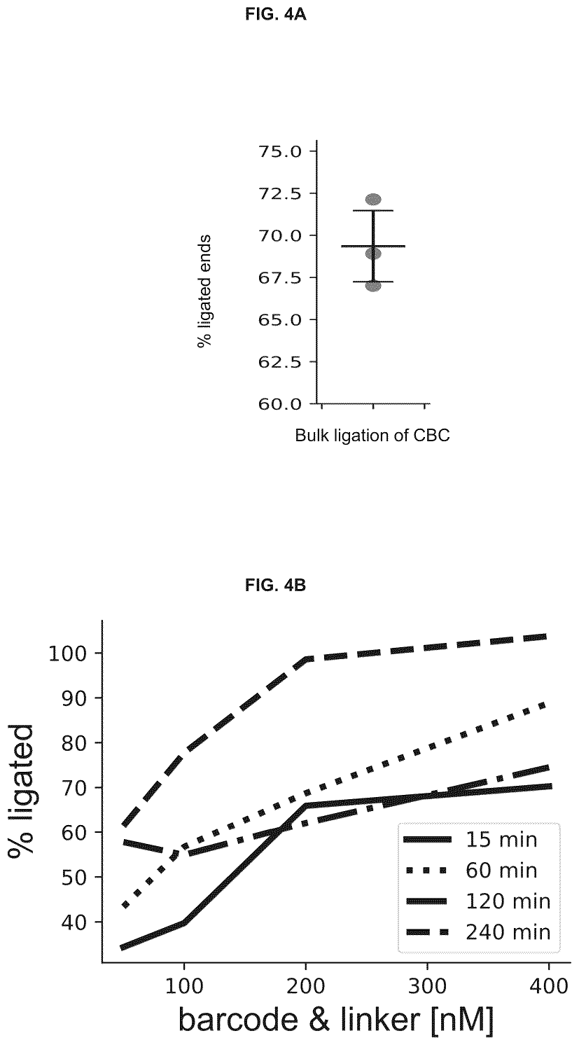

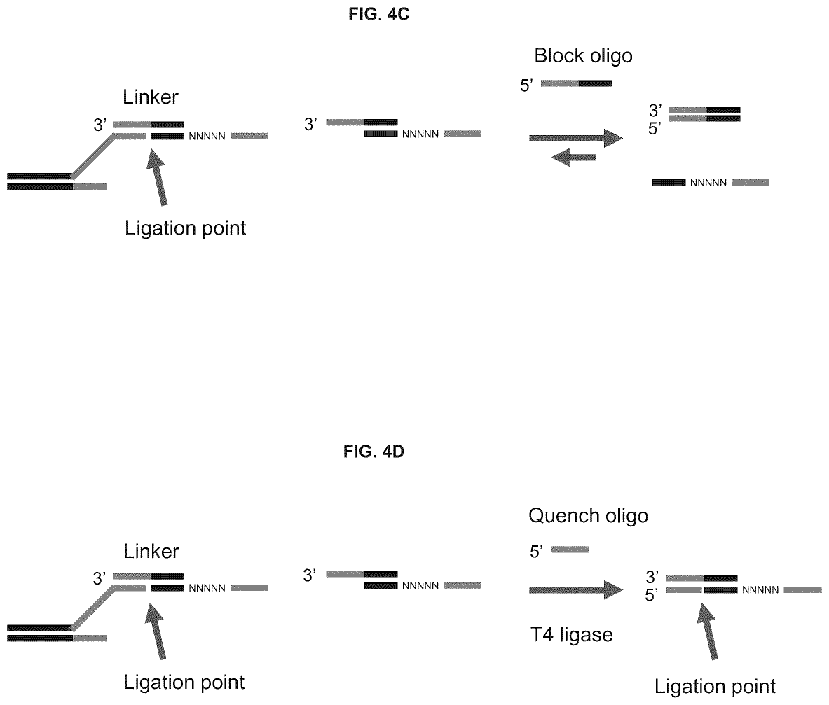

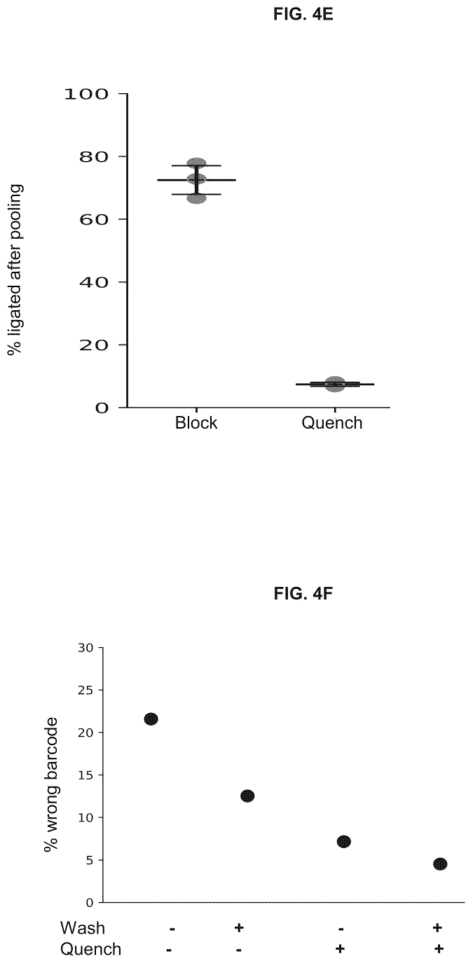

In some embodiments, the hybridization/annealing in the split-pool rounds can further include quenching barcode oligonucleotides identical in sequence to the sequence on the 3′ end of the probe (round 1 barcoding) or the 3′ end of the first barcode universal region (round 2 barcoding). This short quenching barcode oligonucleotide hybridizes to the barcode/linker hybrid in place of the intended probe, thus blocking the barcode/linker hybrid from participating in the typical barcoding ligation reaction (see, e.g., FIG. 4D). This quenching barcode oligonucleotide can also be ligated to the barcode further inhibiting it from participating in future reactions. This is opposed to blocking the linker strand which displaces the linker strand from the barcode strand which inhibits the barcode strand from participating in ligation because its predominant form will be a single stranded oligonucleotide.

In some embodiments, 2, 3, 4, 5, or more rounds of split-pooling is performed, wherein in each of the rounds, the solution comprising the long probes is aliquoted into a plurality of vessels, the vessels containing the ds barcoding oligonucleotide, wherein the barcoding sequence of the ds barcoding oligonucleotide is specific for the vessel in which it resides. The ds barcoding oligonucleotide is annealed to the long probe, or in subsequent rounds a polynucleotide comprising the long probe sequence plus any previously ligated ds barcoding oligonucleotide sequences, and then ligated in the vessels. The overhang sequence of the ds barcoding oligonucleotide that is not annealed to the long probe is selected so that future rounds of split-pooling or subsequent amplification step allow for annealing of subsequent ds barcoding oligonucleotides or universal adapter sequences, respectively.

In the last round of split pooling, an amplification reaction (e.g., PCR) is performed in the vessels using primers that anneal to either end of the long probe barcoded polynucleotide. The primers will use one of end sequence of the end probe itself and one end sequence added by addition of the barcoding sequences, resulting in an amplicon that has universal sequences at either end, for example that can be used as sequencing adapter. One or both of the primers can introduce a further vessel-specific barcoding sequence to the amplicon if desired. In some embodiments, one primer introduces P5 (AATGATACGGCGACCACCGAGATCTACACTCTTTCCCTACACGA; SEQ ID NO:1) and read 1 (ACACTCTTTCCCTACACGACGCTCTTCCGATCT; SEQ ID NO:2) sequences and the other primer introduces P7 (CAAGCAGAAGACGGCATACGAGAT; SEQ ID NO:3) and read 2 (; SEQ ID NO:4) sequences. Sec, e.g., FIG. 1D.

As desired, the resulting amplicons, e.g., cell-specific barcoded long probe polynucleotides, can be nucleotide sequenced. In some embodiments next-generation sequencing (NGS) is used. For example, in some embodiments, massively parallel sequencing is used. Non-limiting examples of next-generation sequencing methods are single-molecule real-time sequencing, ion semiconductor sequencing, pyrosequencing, sequencing-by-synthesis, sequencing-by-ligation, and chain termination. Sequencing adapters for flow cell attachment may comprise any suitable sequence compatible with next generation sequencing systems, e.g., 454 Sequencing, Ion Torrent Proton or PGM, and Illumina X1O. Non-limiting examples of sequencing adapters for next generation sequencing methods include P5 and P7 adapters suitable for use with Illumina sequencing systems; TruSeq Universal Adapter; and TruSeq Indexed Adapter. In some embodiments, a sequencing adapter can be used to enrich, e.g., via amplification, such as polymerase chain reaction (PCR), for polynucleotides comprising the adapter sequence. Sequencing adapters can further comprise a barcode sequence and/or a sample index sequence.

Also provided herein are reaction mixtures generated from the methods as described herein. In some embodiments, the reaction mixture can comprise, for example, fixed and permeabilized cells and single-stranded (ss) DNA probe pairs (as described herein) diffused into the cells. For example, in some embodiments, at least some of the ss DNA probe pairs anneal to RNA in the cells. In some embodiments, the probe pairs comprise a 5′ binding probe and a 3′ binding probe, wherein the 5′ binding probe and the 3′ binding probe anneal to adjacent sequences in a target RNA. In some embodiments, the 5′ binding probe comprises a 5′ universal sequence that does not anneal to the target RNA and a 3′ RNA annealing sequence and the 3′ binding probe comprises a 5′ phosphorylation, a 5′ RNA annealing sequence, and a 3′ adapter sequence that does not anneal to the target RNA. Reaction mixtures can further include, for example, a ligase.

In some embodiments, reaction mixtures are provided that comprise fixed and permeabilized cells and long probes formed by ligation of single-stranded (ss) DNA probe pairs as described herein. In some embodiments, the reaction mixture is a bulk solution. In some embodiments, the solution comprising the reaction mixture is in a plurality of vessels (e.g., at least 10, 20, 50, 100, or more vessels). The reaction mixtures in the vessels can further comprise double-stranded (ds) barcoding oligonucleotides comprising (i) a first (3′ or 5′) overhang sequence and (ii) a central double-stranded sequence having a vessel-specific barcode sequence and (iii) a second (3′ or 5′) overhang sequence, wherein the first overhang anneals or is capable of annealing to (i.e., is reverse complementary to) the 3′ end or the 5′ end of the long probe sequence.

Also provided are kits for performing the methods described herein. The kits can include any one or combination of reagents described in the context of the methods. For example, the kits can comprise one or a plurality of (e.g., at least 10, 50, 100 or more different) single-stranded (ss) DNA probe pairs as described herein. In some embodiments, the kits further comprise a ligase, e.g., for ligating the single-stranded (ss) DNA probe pairs when they anneal to adjacent sequences on an RNA. In some embodiments, the ligase is a PBCV-1 DNA ligase. In some embodiments, the kit comprises one or more cell fixation or permeabilization agent. In some embodiments, the kit comprises a plurality of vessels, optionally containing or coming with a plurality of double-stranded (ds) barcoding oligonucleotides as described herein. In some embodiments, the kit comprises the primers for generating the final amplicon product that can be sequenced.

### Example

We have developed the Hybridization of probes to RNA targets followed by Sequencing (HybriSeq) method for single-cell RNA profiling, which utilizes in situ hybridization of multiple probes for targeted transcripts, followed by split-pool barcoding and sequencing analysis of the probes. We have shown that HybriSeq can achieve high sensitivity for RNA detection with multiple probes and profile RNA accessibility. The utility of HybriSeq is demonstrated in characterizing cell-to-cell heterogeneities of a panel of 95 cell-cycle-related genes and the probe-probe heterogeneity within a single transcript.

This method involves in situ hybridization of multiple split single strand DNA (ssDNA) probes to one or many target RNAs in fixed and permeabilized cells (FIG. 1A), ligating these split probes hybridized to the RNA to ensure specificity (FIG. 1B), ligating a unique cell barcode to the hybridized probes via 2 rounds of split-pool barcoding followed by an indexed PCR (FIG. 1C), and sequencing the ligated probe-barcodes (FIG. 1D). This method can sensitively detect transcripts in a targeted fashion without the need for microfluidics. We demonstrate the utility of this method by profiling the cell-cell heterogeneity in an asynchronous immortalized cell line.

### Material and Methods

### HybriSeq Split Probe Design

HybriSeq ssDNA probes are composed of five regions split into two probes as follows from 5′ to 3′:
(Left probe) 20 nt priming region which is a partial Illumina Nextara read 2 or a different universal priming region.
(Left probe) 30 nt left probe targeting region.
(Right probe) 30 nt right probe targeting region. The first two bases are either A or T. SplintR ligase has higher efficiency when C or G are not in the first two bases of the ligation site.
(Right probe) 8 nt random UMI sequence
(Right probe) 20 nt round 1 ligation handle

A probe design pipeline was adapted from Moffitt et al. (20). With minor changes. For calculating gene and isoform level specificity of probes our pipeline only considers the center 30 nt of the targeting region (last 15 nt left probe+first 15 nt of right probe) and does not directly consider melting temperature as a parameter when selecting probes but considers CG content.

Probes were obtained from IDT (Integrated DNA Technologies) in the 50 nmole oPools format or individually as single probes ordered as DNA oligos.

Right side probes were 5′ phosphorated with T4 Polynucleotide Kinase (NEB). Probes were then column cleaned with ssDNA/RNA Clean & Concentrator (Zymo D7010) and quantified. Left side probes were added at an equal molar concentration and used in hybridization.

### HEK293 Cell Culture

HEK293 cells were cultured in DMEM+10% FBS & 1% Penicillin-Streptomycin. Cells were washed twice with 1×PBS, then detached by incubating 2-5 min at room temperature with 3 ml of 0.25% Trypsin. Once cells were detached, they were added to 7 Ml of media with 10% FBS. In cell mixing experiments, cells were combined at the desired concentrations at this step.

### Fixation

Cells were centrifuged for 3 min at 500 g at 4° C. Cells were washed in 1 Ml of 1×PBS. The cells were then passed through a 40 μm strainer into a 15 Ml falcon tube and counted. Cells were centrifuged for 3 min at 500 g at 4° C. Cells were resuspended in 0.5 ml/million cells of 4% freshly prepared formaldehyde solution in 1×PBS. Cells were fixed for 30 min at room temperature under gentle agitation. Cells were centrifuged for 3 min at 500 g at 4° C. and washed 2 times in 1×PBS. The cells were then passed through a 40 μm strainer into a 15 Ml falcon tube and counted.

### Hybridization & Ligation

Cells were resuspended in Hybridization buffer (30% formamide, 1% BSA, 0.5% tween 20, 2×SSC, 40 U/ml Rnasin) for 10 min at 37° C. under gentle agitation. Cells were centrifuged for 3 min at 500 g at 4° C. Cells were resuspended in Hybridization buffer with probes at 10 Nm/probe. Cells were incubated at 37° C. for 18-24 h with gentle agitation. Cells were then washed in wash buffer (20% formamide, 0.5% tween 20, 4×SSC, 40 U/ml Rnasin) two time at 37° C. for 5 min. Cells were washed in ligation buffer (1×T4 DNA Ligase Reaction Buffer (NEB), 0.4 Mm ATP, 40 U/ml Rnasin) and then resuspended in ligation buffer plus 2 Um SplintR Ligase (NEB). Cells were incubated for 1 h at 37° C. with gentle agitation.

### Preparing Oligos for Ligations

The first and second barcoding steps consist of a ligation reaction. Each round uses a different set of 96 well barcoding plates. Ligation rounds have a universal linker (Supplementary table S5) strand with partial complementarity to a second strand containing the unique well specific barcode sequence added to each well (Supplementary table S6,S7). These strands were annealed together prior to barcoding to create a DNA molecule with three domains: a 15 nt 5′ overhang that is complementary to the 15 nt 3′ overhang present on the right-side probe, a well-specific barcode sequence, and a 15 nt 3′ overhang complementary to the 5′ overhang present on the next barcode molecule to be subsequently ligated. For the second-round barcodes, the 3′ overhang acts as a universal priming region to which the third round well specific primer can anneal and extend in a PCR. Barcode strands (IDT) for the ligation rounds are added to 96 well plates and their 5′ ends phosphorylated with T4 Polynucleotide Kinase (NEB). After 5′ phosphorylation, equal molar amounts of linker strand are added to each well making the final concertation 5.4 Um. Oligos for ligation are annealed by heating plates to 95° C. for 2 minutes and cooling down to 20° C. at a rate of −0.1° C. per second. For ligation reactions, 2.31 ul of barcode/linker oligos are added to 96 well plates to which cell can be added.

### Cell Barcoding

After probe ligation cells were counted and added to the ligase buffer (1×T4 DNA Ligase Reaction Buffer (NEB), 0.4 Mm ATP, 40 U/ml Rnasin, 0.5% tween 20, 1% BSA, 8 (106) U/ml T4 ligase) so that the final volume was 1.1 ml at a 22,000 cells/ml. Cells were passed through a 40 μm strainer. 22.69 ul of cells in ligase buffer were added to each well of 48 wells of a 96 well protein low bind plate which had 2.31 ul of barcode 1 and linker 1 oligos already in each well. Cells were mixed by gently pipetting up and down. Plates were sealed and incubated at 25 C for 2 h. 2 ul of 62.5 Um quenching oligo 1 (Supplementary table S5) were added to each well and mixed by pipetting. Plates were sealed and incubated at 25 C for 30 min. 25 ul of barcode wash buffer (50 Mm EDTA, 0.5% tween 20) was added to each well and incubated for 10 min. Cells from all 48 wells were pooled into a single 5 ml low bind Eppendorf tube. Cells were centrifuged for 3 min at 500 g at 4° C. Cells were washed two times in barcode wash buffer (+5 Um quenching oligo 1) and then washed in ligase buffer (+5 Um quenching oligo 1, −T4 ligase). Cells were resuspended in 1.1 ml ligase buffer (+5 Um quenching oligo 1) and passed through a 40 μm strainer. 22.69 ul of cells in ligase buffer (+5 Um quenching oligo 1) were added to each well of 48 wells of a 96 well protein low bind plate which had 2.31 ul of barcode 2 and linker 2 oligos already in each well. Cells were mixed by gently pipetting up and down. Plates were sealed and incubated at 25 C for 2 h. 2 ul of 62.5 Um quenching oligo 2 were added to each well and mixed by pipetting. Plates were sealed and incubated at 25 C for 30 min. 25 ul of barcode wash buffer was added to each well and incubated for 10 min. Cells from all 48 wells were pooled into a single 5 ml low bind Eppendorf tube. Cells were centrifuged for 3 min at 500 g at 4° C. Cells were washed two times in barcode wash buffer (+5 Um each of quenching oligo 1 & 2) and then resuspended in ice cold 1×ThermoPol reaction buffer (NEB) cells were passed through a 40 μm strainer and counted. Cell concentration was normalized to 23,000 cells/ml in cold ThermoPol reaction buffer. 115 cells were dispensed into 8 wells of a strip tube. 20 ul of PCR solution (1×KAPA HiFi HotStart ReadyMix (final concentration) and forward primer) with well specific round 3 reverse primers added to each well so that the final concentration of each primer was 0.4 Um. PCR thermocycling was performed as follows: 95° C. for 30 sec, then 20 cycles at 95° C. for 30 seconds, 55° C. for 30 seconds, 72° C. for 30 seconds, followed by a final extension at 72 C for 30 seconds.

### Library Preparation and Sequencing

Round 3 PCR reactions were centrifuged at full speed for 1 min to pellet cells. All round 3 PCR reaction solution was removed, pooled, and column purified with the Zymo DNA clean & concentrator kit (Zymo 11-305). Purified libraries were analyzed on an Agilent TapeStation Systems (D1000 kit) to check for the correct size. If the predominate band was the correct size (252±2 bp or 232±2 bp depending if the left probe included a partial read 2 sequence) and was <90% of the library the purified PCR product was run on a 2% agarose (TBE) electrophoresis gel (200V 20 min) and the correct size band was cut out and extracted from the agarose with the Zymo Gel recovery kit (Zymo D4002). We observe that libraries that contained left probes containing the non read 2 priming regions produced some nonspecific amplification requiring size selection purification. The purified pooled round 3 DNA product was placed into a final limited cycle PCR to add Illumina sequencing adaptors. The adapter addition PCR reaction was as follows: 0.5 ng DNA from pooled round 3 PCR product, 0.4 Um P7 forward primer, 0.4 Um P5 reverse primer and 1×KAPA HiFi HotStart ReadyMix. PCR thermocycling was performed as follows: 95° C. for 30 sec, then 10 cycles at 95° C. for 30 seconds, 55° C. for 30 seconds, 72° C. for 30 seconds, followed by a final extension at 72 C for 30 seconds. The PCR reaction was removed and purified with a 0.8× ratio of SPRI beads to generate an Illumina-compatible sequencing library.

### Illumina Sequencing

15 Pm libraries were sequenced on a MiSeq (Illumina) using a 150 nucleotide (nt) V3 kit in paired-end format. Read 1 (75 nt) covered the cell barcode and read 2 (75 nt) covered the probe and UMI.

### Rnase H Specificity

After non-split probe hybridization and washing, cells were resuspended in Rnase H reaction buffer containing 20 U/ml of Rnase H enzyme (NEB M0297S). Cells were incubated for one hour at 37° C. with gentle agitation. Released probes were quantified with sequencing or qPCR.

qPCR

qPCR was performed on probes released from cells via Rnase H release or heat release that were purified with spin columns (Zymo ssDNA/RNA clean & concentrator). 1 ul of purified samples were loaded into each reaction of a qPCR with 0.3 Um primers according to manufacturer's instructions using Maxima SYBR Green qPCR Master Mix (Thermo Fisher Ref K0222).

### HybriSeq Computational Pipeline

We constructed a pipeline to analyze HybriSeq data by taking raw sequencing reads and constructing a count matrix (counts per probe per cell). Briefly, we identify real barcodes, identify probe targeting regions with correct ligation, remove duplicates using UMIs, and filtered out reads not containing barcodes or probe targeting regions. Detailed key steps were as follows:

From the demultiplexed FASTQs generated by the Illumina analysis software we filtered out reads not containing common regions contained in barcode one, two, and three in the correct location.

To determine the unique barcode, a whitelist of each round of barcode sequences were constructed including barcodes within a hamming distance of two. With this list, barcode sequences for each round of split pool indexing were determined from read 1. From this a unique cell barcode was constructed. Reads for which no barcode could be found were excluded.

To determine the targeting region from read 2, a whitelist of each probe was constructed including probe sequences within a hamming distance of two. With this list, both left and right side probe targeting regions were determined. Reads containing targeting regions not predicted to be adjacent were excluded. From read 2 we also extracted the 8 bp simple UMI included on the right side probe.

We constructed a data frame of reads that included the unique cell barcode, probe targeting region, and simple 8 bp UMI. We then collapsed duplicate reads by considering a combined UMI which contained the 8 bp simple UMI, the unique cell barcode, and the probe targeting region.

We generate a count table of UMIs per probe per unique cell barcode or UMIs per transcript per unique cell barcode.

To determine which unique cell barcodes were associated with real cells a threshold for UMIs/cell was calculated by taking 10% of the 99th percentile of the top set of unique cell barcodes equal to the number of expected cells and considering a doublet rate of ˜5% or visually setting the threshold at the first knee of the cell rank-UMI plot. We note that when only considering lowly or highly variably expressed transcripts that inclusion of probes targeting moderately and stably expressed transcripts can help set a threshold.

The Scanpy library in Python was used for all standard single cell analysis.

### Mixing/Single-Cell Purity Experiment

Two HEK293 cell lines, each containing a specific transcript (mNeonGreen1-10 (mNG) and GFP1-10 (GFP)) were subjected to the standard HybriSeq protocol. Probes targeting mNG and GFP (Supplementary table S1) were added to the probe mixture during the hybridization step. At the PFA fixation step, equal concentrations of each cell line were mixed.

### Probe Tiling Analysis

Each transcript was analyzed independently only considering probes targeting that transcript. Probe counts for each cell were normalized so that the total sum of all normalized counts in each cell was equal to the median UMIs/cell of the cell population. This was done to account for differences in expression levels between cells. The average relative counts were taken for each probe and plotted as a trace for all cells or pseudo bulk clusters. The standard deviation was calculated for cell populations for each probe.

### Measurement Variability Model and Simulation

To model measurement noise associated with sampling a specific transcript in a cell we started off by making a few assumptions.
Sampling of a transcript in a cell can be modeled with a Poisson distribution.
The probability of capturing a transcript or probe is the same for all probes targeting the same transcript or priming events.
The background signal from random probe ligation is minimal and can be assumed to be negligible.
Probe binding a transcript does not influence different probes binding the same transcript.
Probes are hybridized at a saturating concentration.
The underlying cell-cell heterogeneity can be modeled as a constant value of standard deviation and is not dependent on the number of probes used.
All cells have the same efficiency of detection for the same transcript.

### To Model Single Cell Transcript Measurement Variability:

Let N be the number of specific transcripts in a cell, n be the number of detection chances per transcript in a cell, e be the efficiency at which n is successfully detected, and C be the number of counts or UMIs for a specific transcript. If we assume that N is Poisson, the variability associated with counts C is equal to the mean of C and we define measurement noise as the standard deviation of the measurement C:

$$C = ( N ) ( n ) ( e ) , \qquad ( 1 , 2 )$$

$$Noise\_C = σ = √ ( ( N ) ( n ) ( e ) )$$

Taking the ratio of the counts C to the noise associated with C we get the signal to noise ratio (SNR)

$$SNR = √ ( ( N ) ( n ) ( e ) ) \qquad ( 3 )$$

For a population of cells, C will scale linearly with n. If we define expression, M, as C normalized to the number of probes used to make the measurement, expression is given by:

$$M = C / n = ( N ) ( e ) ( n ) / n = ( N ) ( e ) , \qquad ( 4 , 5 )$$

$$Noise\_M = √ ( ( N ) ( n ) ( e ) ) / n + b = √ ( M / n ) + b$$

Here we assume that that the contribution to noise in the expression measurement from the biology is independent of the number of probes used to make the measurements and can be defined as constant b.

The expression SNR is then given by the ratio of M to Noise associated with M:

$$SNR\_M = M / ( √ ( M / n ) + b ) \qquad ( 6 )$$

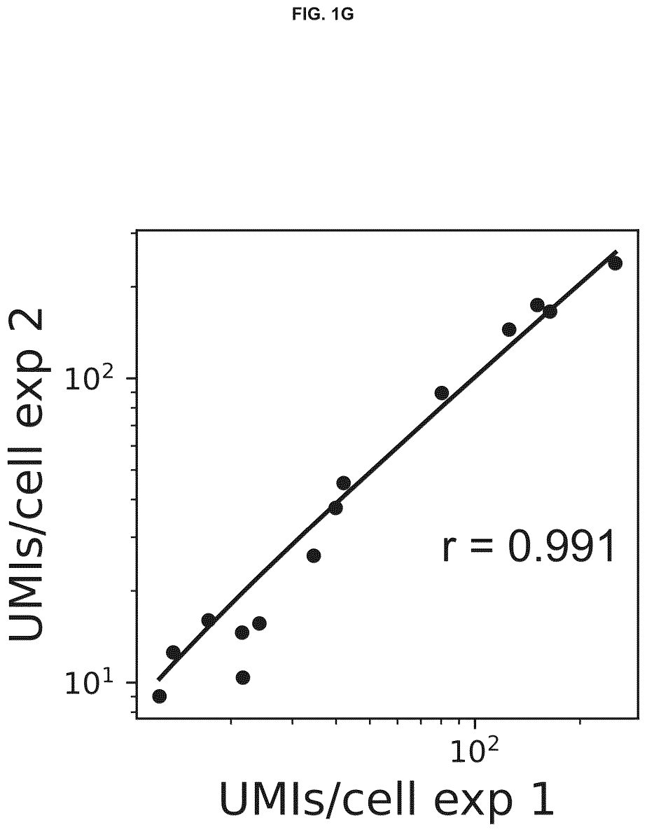

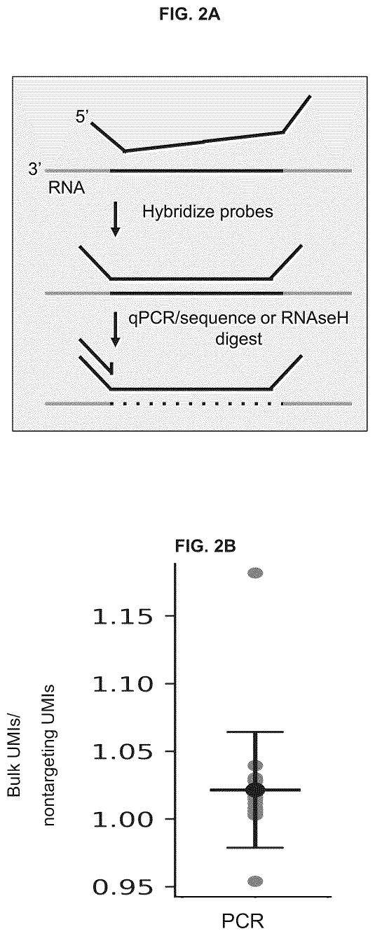

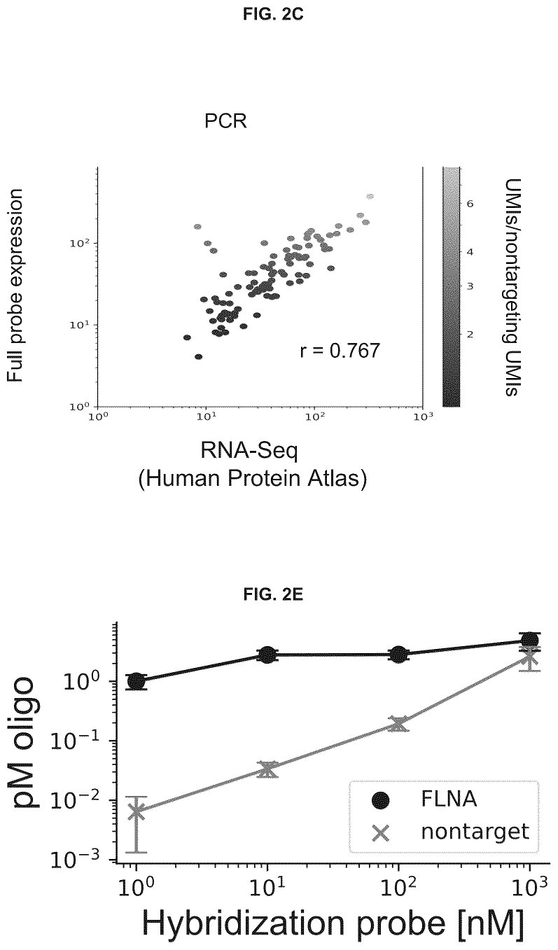

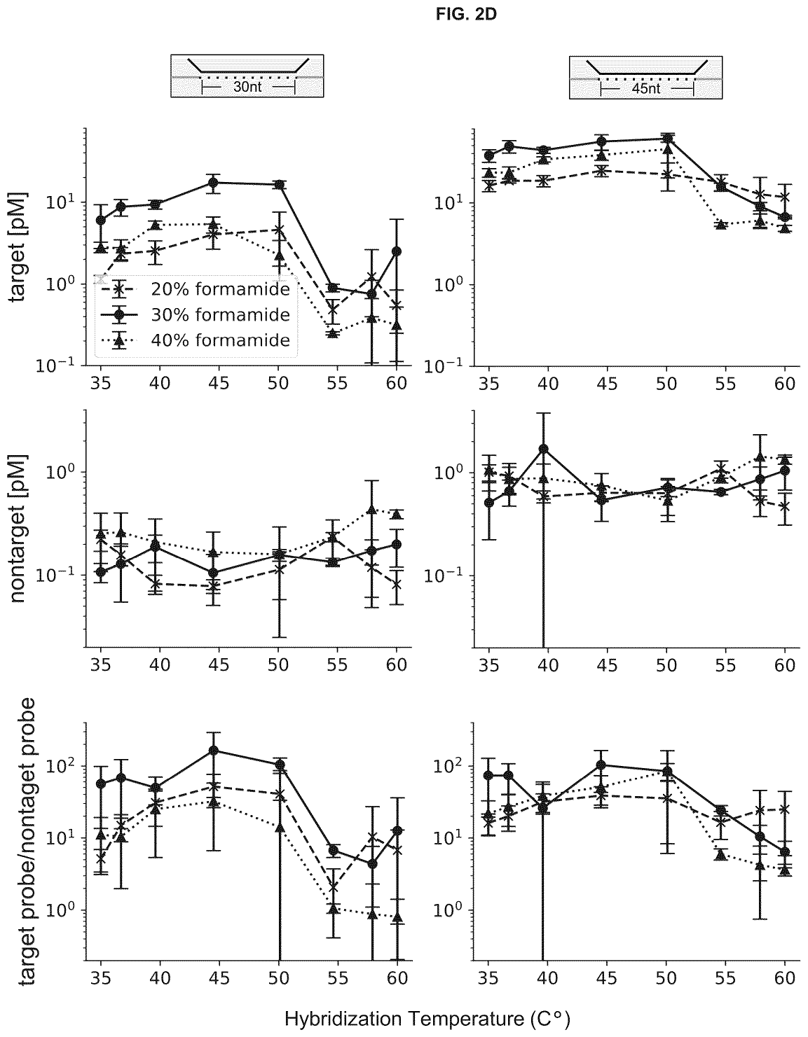

For the simulations in FIG. 2 we experimentally determine M by taking the slope of the line fit to the UMI/cell—probe number plot. This slope is the number of counts you would expect to gain for each additional probe included in the analysis.

We then non-linearly used least squares to fit the function for Noise of M to the standard deviation of M as a function of the number of probes used to make the measurement keeping M constant from the experimentally determined M and only fitting the model by optimizing b.

### Calculation of Signal, Standard Deviation, and SNR for Multiple Probes

Total probe counts for each cell were normalized so that the total sum of all normalized counts in each cell was equal to the total median UMIs/cell of the cell population. This was done to account for differences in expression levels between cells as the goal is to gain an understanding of the measurement associated variation and not necessarily the underlaying inherent biological variation. To calculate the average signal, or counts, for each number of probes considered (n), a random set of probes was chosen without replacement and the number UMIs/cell was calculated along with a standard deviation for each n. To calculate the SNR, the ratio of average expression (UMIs/cell/n) to the standard deviation of expression was calculated for all n. This was repeated 10,000 times, randomly sampling the set of probes used to make the measurement and the average and standard deviations of these calculations were plotted.

### Results

### Development and Validation of HybriSeq

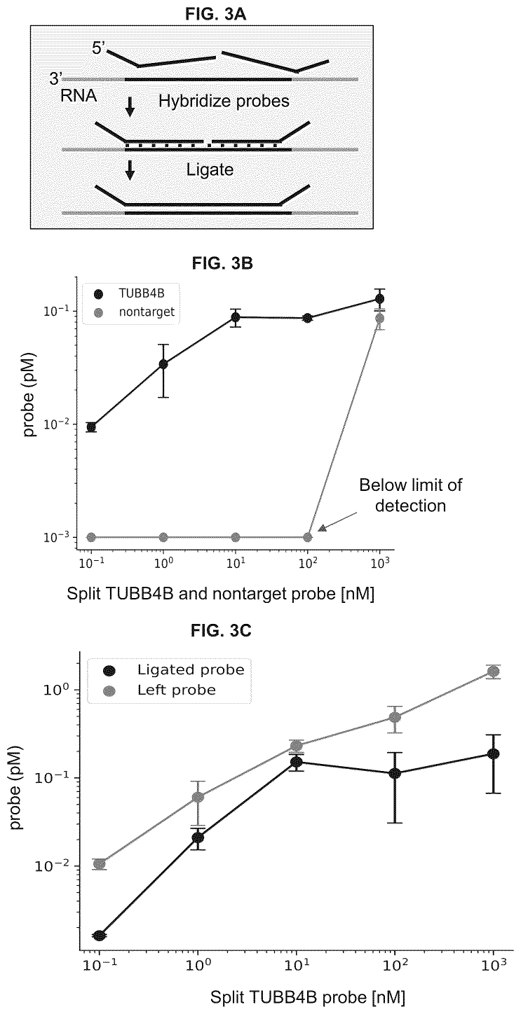

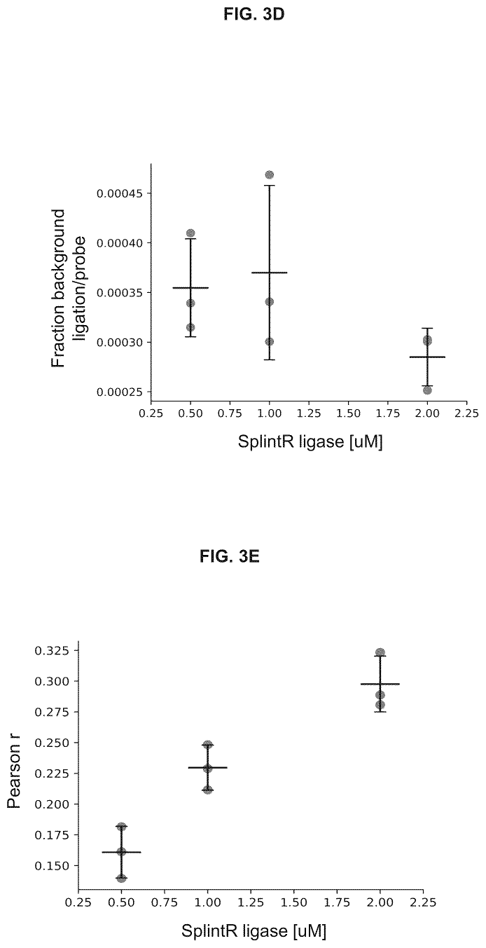

To establish a method for efficient hybridization and recovery of ssDNA probes to target RNAs with low nonspecific binding, we performed in situ hybridization in fixed and permeabilized HEK293 cells in suspension and quantified the efficiency and specificity of probe recovery by sequencing (FIG. 2A). We found that ssDNA probes have non-negligible nonspecific binding to the cells, which can contribute to background signal (FIG. 2B). We tried to improve the specificity by releasing hybridized but not nonspecifically bound probes using RNAes H digestion of the cells (FIG. 2C), but the signal/background ratio was still low even after optimizing hybridization conditions (FIG. 2D). Therefore, we adopted a method similar to LISH (13), splitting the probe into two parts and ligating hybridized pairs using SplintR ligase that acts on DNA-RNA hybrids (FIG. 1A, B). Bulk level qPCR measurements of ligated probes in cells showed that with ligation it is possible to saturate the probe signal from a high expression transcript (FIGS. 3B-C) and achieve a specificity >99% (FIG. 3B).

To enable single cell analysis, we adapted the split-pooling method (11) to uniquely label the probes in individual cells with cell specific barcodes. In 96-well plates, hybridized and ligated probes are labeled with well-specific barcodes via ligation on the 3′ end in two rounds of split and pool procedures followed by a third round of barcoding by PCR with well specific primers (FIG. 1C). Depending on the path a cell takes through this procedure, all the probes in that cell will have one of 884,736 possible unique cell barcodes (CBC), of which <5% are utilized to avoid excessive CBC collision. Different from previous split-pooling methods, our main challenge is the mixing of barcodes between cells, which can arise from inefficient ligation before pooling, excess ligatable barcode oligos in subsequent ligation reactions, and priming of incompletely ligated species. We screened a variety of barcode ligation and washing/quenching conditions in bulk with qPCR. We found that long ligation times and high barcode oligo concentrations are needed for efficient barcode ligation (FIG. 4B). Additionally, we found that quenching barcode oligos as opposed to blocking linker strands resulted in less barcode hopping and that washing away excess barcodes after each ligation step led to significantly less barcode hopping (FIG. 4C-F).

To investigate the performance of HybriSeq at the single-cell level, we designed a set of probes (5-6 probes per transcript) targeting mNeonGreen1-10 (mNg) and GFP1-10 (GFP) transcripts. Using human embryonic kidney 293 (HEK293) cells stably expressing either mNg or GFP at a variable range of expression levels (14) we profiled these transcripts with HybriSeq and sequenced libraries to a median per cell saturation of 74% (3990 reads per cell) and observed a total of 691 cells (921 cells expected) and a median of 557 UMIs/cell (Unique Molecular Identifier). To determine the single cell purity of HybriSeq, we performed a cell mixing experiment of the mNG and GFP cells in equal proportions. We observed that 2.6% of CBC contained multiple probes from both mNG and GFP suggesting a doublet rate of 5.2% (FIG. 1E) (from a 50/50 mix of cells, half of the doublets will arise from two cells with the same CBC). UMI counts per probe per cell for three highly expressed transcripts (Supplementary table S2) were highly correlated between biological replicates with a Pearson's R>0.99 (FIG. 1G). A median of 99.6% of reads for each CBC were specific to either mNG or GFP probes. These data suggest HybriSeq libraries have a high level of single-cell purity and reproducibility. This multiple rate is higher than the expected multiple rate of 2.45%. This is most likely due to cell clumping, ambient probes, or RNA leaking from cells. While nonzero this is lower than most droplet-based approaches.

HybriSeq specificity arises from both specific hybridization of ssDNA to transcripts and from the ligation of two adjacent probes hybridized. To evaluate the specificity of HybriSeq we looked at reads in the library that contained left probe and right probe targeting regions not predicted to be adjacent to each other. We compared the amount of these nonspecific ligation events to the specific and correctly ligated events. mNG probes gave >400,000-fold higher signal than nonspecific ligation events with a median 302 UMIs per cell, and GFP probes gave >1,000,000-fold higher signal then nonspecific ligation events with a median 869 UMIs per cell. The average number of nonspecific UMIs per cell was 0.00023. This result suggests that HybriSeq is highly specific.

### Quantitative Accuracy of HybriSeq

To demonstrate the profiling of a panel of RNAs using HybriSeq, we constructed a set of probes targeting 95 transcripts (2-4 probes per target) associated with the cell cycle (Supplementary table S3). These transcripts range in bulk expected expression of 5-355 Transcripts per million (TPM) in HEK293 cells (15). Using this set of probes, we performed HybriSeq for an asynchronous population of HEK293 cells. The resulted bulk expression values correlate well with published bulk RNA-Seq data (15). (r=0.7) (FIG. 1F). UMI counts per cell for probes targeting the same transcript correlate well, with 72% of same transcript probe pairs having an Pearson's R>0.8. To determine the effect on measurement precision if fewer probes per transcript were used, we subsampled the number of probes used to calculate a Pearson correlation coefficient. The use of 3-4 probes per transcript was optimal, while less precise results were seen when 1-2 probes were sampled.

Next, we quantify the relationship between measurement noise and probe number using a simple mathematical model. In many high throughput scRNAseq methods, an individual transcript frequently “dropped out” in the digital gene expression count, making the measurement of lowly expressed transcripts excessively noisy. This issue is in part the result of the nature in which transcripts are sampled by a single priming event at the poly-A tale of transcripts, followed by losses in subsequent reverse transcription and capturing steps. With only one chance to detect a transcript, the probability of detecting that transcript becomes a binomial trial with exactly two outcomes (detected and not detected). The use of multiple probes in HybriSeq, on the other hand, serves as a linear amplification of the transcript before the lossy detection. We approximate the detection of a specific transcript as a Bernoulli trial and modeled with Poisson sampling. In this case, the signal to noise ratio (SNR) in a typical scRNAseq measurement is approximately the square root of the product of the molecules present and the efficiency of capture. Applying this model with the best detection efficiency reported of 45% and a SNR threshold of 2 the lowest number of molecules reliably detected is 8. With the more typical detection efficiency of 10% this number is closer to 40 molecules. Now for the same model with a linear amplification factor as in HybriSeq and an average detection efficiency for a single probe of 20%, a similar or better lower limit of detection can theoretically be accomplished with >2 probes, consistent with our subsampling analysis Moreover, near single-molecule sensitivity can be achieved when >10 probes are used.

To test our model, we constructed a set of probes completely tiling six transcripts with an expression level from 15-165 TPM in HEK293 cells. These transcripts are expected to only have one isoform expressed that does not have expected variation during the cell cycle, which is the main source of heterogeneity in a monoculture cell system. Our model predicts that for a given transcript/cell value and efficiency of capture the number of UMIs/transcript will increase in a linear fashion with respect to the number of probes subsampled from the measurement, the standard deviation of the expression (UMIs/transcript/unique probes) will fall off 1/square root of the probe number, and the SNR will increase as a function of the square root of the probe number. We observed for all transcripts probed that our simple model explains the trends in the SNR. For all but one (NEFH) of the transcripts tested we were able to achieve a SNR>2 with fewer than 6 probes. In fact, near single-molecule/cell sensitivity can be achieved when >20 probes are used (e.g. SCAF8 and ARL5B when summing up all probes.

Our transcript tiling results also reveal probe-to-probe variabilities that cannot be explained by CG content nor probe specificity. In particular, we observed that certain probes are underrepresented in the sequencing readout relative to the average probe number for a specific transcript or hardly represented at all. For EIF2S2 (ENSG00000125977) the 3′ half of the transcript has very few UMIs associated with it. We found that this region is mostly composed of the 3′ untranslated region (UTR). While not as pronounced, this depletion is also seen in GHITM (ENSG00000165678) and NEFH (ENSG00000100285. In contrast, SCAF8, ARL5B, and MARVELD1 showed much more uniform probe occupancy throughout the length of the transcript. Within the cell population profiled, we also observed elevated cell-to-cell heterogeneity in occupancy for a subset of probes. These differences in probe occupancy may be attributed to differentially regulated RNA processing, RNA-protein interactions, secondary structures, etc.

Probing Cell-to-Cell Heterogencities with HybriSeq

A monoculture of proliferating cells will have cell-cell transcriptional heterogeneity due to the asynchronous progression through the cell cycle. To demonstrate the ability for HybriSeq to characterize such heterogeneities, we analyzed the HybriSeq data set which probes the 95 cell-cycle-related transcripts. Dimensionality reduction was performed on the cell gene matrix and the resulting UMAP projection was clustered with the Leiden algorithm. The transcripts with the most variable expression used to define the Leiden clusters showed groupings of genes with similar expression profiles that are typically associated with a particular phase of the cell cycle. When transcripts are grouped together based on known association to one of the cell cycle phases, their scaled expression shows a clear transcriptional program. These results suggest that the Leiden clusters represent rough boundaries of cell cycle phases. Because clustering approaches like Leiden are less efficient in assigning a cell state along a continuous axis of variation, we also used an alternative approach by calculating a phase score for each cell based on known cell cycle associated genes. Based on the binned phase scores, clustering the cells into three phases, G1, S, and G2M, shows a more biologically representative clustering than the Leiden clustering. The proportion of each cell type was similar to a previously published single-cell transcriptome analysis of HEK293 cells (16) when only genes with HybriSeq probes were considered. Additionally, the expression distribution profiles of cell binned by phase score show a clearer trend compared to the subtle trend seen with Leiden clustering and the pattern of co-expression in the scaled expression profile is much clearer when grouped by G1, S, G2M clusters. Notably, our HybriSeq results were obtained using an Illumina MiSeq V3 and substantially fewer reads than other whole transcriptome methods, demonstrating that HybriSeq is an affordable approach to targeted single-cell RNA profiling.

### DISCUSSION

Here, we present HybriSeq, a probe-based, microfluidics-free method to sensitively profile a set of targeted RNA in single cells. HybriSeq provides a unique set of advantages that overcome current limitations in scRNAseq approaches. First, by utilizing many probes per transcript HybriSeq offers the ability to confidently detect low expression transcripts by decreasing the measurement noise. Second, because of the targeted and scalable nature of probe-based split-pool methods, HybriSeq can cost effectively profile specific biology in many cells by only including probes for transcripts of interest, which greatly increases the efficiency of sequencing and reduces the cost. Finally, HybriSeq utilizes a split-pool approach to label cells with unique cell barcodes, which eliminates the need for microfluidic devices used in other probe-based single-cell RNA profiling methods (8,9,10). This feature allows for the use of cost effective, off-the-shelf reagents and a simple protocol that is accessible to most users. The unique features of HybriSeq unlock possibilities that were once unattainable with conventional scRNAseq methods. For example, HybriSeq could profile cell-cell heterogeneity in transcript accessibility of regulatory RNA or used to understand cis- and trans-RNA interactions regulating translation. The distinctive features of HybriSeq lies in its ability to accurately quantify RNA expression and accessibility across diverse transcripts, facilitating the study of cellular transcriptional heterogeneity with heightened sensitivity and resolution.

While powerful in its ability to sensitively detect RNA, the sensitivity of HybriSeq and other probe-based single-cell RNA profiling methods is limited by the length of the RNA molecule being measured, which restricts the total number of probe binding sites. This is the case for all in situ hybridization-based approaches and methods utilizing random priming or cDNA fragmentation. For short RNA targets, the number of probes able to hybridize to a transcript could be small even with reduced probe length. A potential workaround to this problem is to use probes with partially overlapped hybridization target regions, as has been utilized in multiplexed FISH methods (7). Moreover, although probe-based methods are efficient in counting transcript copy numbers, they are not designed to sequence the RNA molecule itself, thus rendering it inappropriate for detecting RNA sequence variants or modifications. Last, a limitation for HybriSeq is that probe hybridization and cell barcoding require multiple rounds of washes as well multiple ligation steps. Each of these steps is associated with inefficiency that contributes multiplicatively to decreased sensitivity. Increasing the probe number per transcript could in some cases compensate for these inefficiencies.

Our transcript tiling results have shown probe-to-probe variabilities that cannot be explained by CG content or specificity for transcripts not known to be alternatively spliced in the cells used. For some transcripts, 3′-UTR targeted probes showed lower abundance than those targeting the rest of the transcript. It is known that the UTR of transcripts can be highly structured and interact with regulatory proteins (17, 18, 19). Therefore, RNA-protein interaction, cis- and trans-RNA interactions, and overall molecule accessibility might partially explain these differences in probe reads. Further considering that certain probes show higher cell-to-cell variabilities compared to other probes targeting the same transcript, this pattern of enrichment/depletion may indeed be indicative of underlaying biology pertinent to gene expression regulation and cell-to-cell heterogeneity. In the case of transcripts with alternative splicing, such analysis can still be performed by including probes for introns and across splicing junctions, showcasing the advantage of non-3′-biased detection in HybriSeq. Furthermore, investigation into this phenomenon will also yield useful insights into probe design for FISH-based spatial transcriptomic approaches, which rely on hybridization to make measurements.

### REFERENCES

1. David Osumi-Sutherland, Chuan Xu, Maria Keays, Adam P Levine, Peter V Kharchenko, Aviv Regev, Ed Lein, Sarah A Teichmann (2021) Cell type ontologies of the Human Cell Atlas. Nat Cell Biol. 11, 1129-1135.
2. Madalee G. Wulf, Sean Maguire, Paul Humbert, Nan Dai, Yanxia Bei, Nicole M. Nichols, Ivan R. Corrêa Jr., Shengxi Guan (2019) Non-templated addition and template switching by Moloney murine leukemia virus (MMLV)-based reverse transcriptases co-occur and compete with each other MMLV-type reverse transcriptase template switching. J BIOL CHEM., 48, 18220-18231.
3. Xiannian Zhang, Tianqi Li, Feng Liu, Yaqi Chen, Jiacheng Yao, Zeyao Li, Yanyi Huang, Jianbin Wang (2019) Comparative Analysis of Droplet-Based Ultra-High-Throughput Single-Cell RNA-Seq Systems. Mol. Cell., 73, 1, 130-142.
4. Johannes W. Bagnoli, Christoph Ziegenhain, Aleksandar Janjic, Lucas E. Wange, Beate Vieth, Swati Parekh, Johanna Geuder, Ines Hellmann & Wolfgang Enard (2018) Sensitive and powerful single-cell RNA sequencing using mcSCRB-seq. Nat. Commun., 9, 2937.
5. Valentine Svensson, Kedar Nath Natarajan, Lam-Ha Ly, Ricardo J Miragaia, Charlotte Labalette, Iain C Macaulay, Ana Cvejic & Sarah A Teichmann (2017) Power analysis of single-cell RNA-sequencing experiments. Nat. Methods., 14, 381-387.
6. Elisabetta Mereu, Atefeh Lafzi, Catia Moutinho, Christoph Ziegenhain, Davis J. McCarthy, Adrián Álvarez-Varela, Eduard Batlle, Sagar, Dominic Grün, Julia K. Lau et al. (2020) Benchmarking single-cell RNA-sequencing protocols for cell atlas projects. Nat. Biotechnol., 38, 747-755.
7. Guiping Wang, Jeffrey R. Moffitt & Xiaowei Zhuang. (2018) Multiplexed imaging of high-density libraries of RNAs with MERFISH and expansion microscopy. Sci. Rep., 8, 4847.
8. Jamie L. Marshall, Benjamin R. Doughty, Vidya Subramanian, Philine Guckelberger, Qingbo Wang, Linlin M. Chen, Samuel G. Rodriques, Kaite Zhang, Charles P. Fulco, Joseph Nasser et al. (2020). Proc. Natl. Acad. Sci. U.S.A. HyPR-seq: Single-cell quantification of chosen RNAs via hybridization and sequencing of DNA probes., 117, (52), 33404-33413.
9. Ryan McNulty, Duluxan Sritharan, Seong Ho Pahng, Jeffrey P. Meisch, Shichen Liu, Melanie A. Brennan, Gerda Saxer, Sahand Hormoz & Adam Z. Rosenthal. (2023) Probe-based bacterial single-cell RNA sequencing predicts toxin regulation. Nat. Microbiol., 8, 934-945.
10. Amanda Janesick, Robert Shelansky, Andrew Gottscho, Florian Wagner, Morgane Rouault, Ghezal Beliakoff, Michelli Faria de Oliveira, Andrew Kohlway, Jawad Abousoud, Carolyn Morrison (2022) High resolution mapping of the breast cancer tumor microenvironment using integrated single cell, spatial and in situ analysis of FFPE tissue. Biorxiv https://doi.org/10.1101/2022.10.06.510405
11. Alexander B Rosenberg, Charles M Roco, Richard A Muscat, Anna Kuchina, Paul Sample, Zizhen Yao, Lucas T Graybuck, David J Peeler, Sumit Mukherjee, Wei Chen et al. (2018) Single-cell profiling of the developing mouse brain and spinal cord with split-pool barcoding. Science., 360 (6385), 176-182.
12. Sanjay R Srivatsan, José L McFaline-Figueroa, Vijay Ramani, Lauren Saunders, Junyue Cao, Jonathan Packer, Hannah A Pliner, Dana L Jackson, Riza M Daza, Lena Christiansen et al. (2020) Massively multiplex chemical transcriptomics at single-cell resolution. Science. 367 (6473) 45-51.
13. Joel J Credle, Christopher Y Itoh, Tiezheng Yuan, Rajni Sharma, Erick R Scott, Rachael E Workman, Yunfan Fan, Franck Housseau, Nicolas J Llosa, W Robert Bell et al. (2017) Multiplexed analysis of fixed tissue RNA using Ligation in situ Hybridization. Nucleic Acids Res., 45, (14), e128.
14. Siyu Feng, Sayaka Sekine, Veronica Pessino, Han Li, Manuel D. Leonetti & Bo Huang (2017) Improved split fluorescent proteins for endogenous protein labeling. Nat. Commun. 8, 370.
15. Max Karlsson, Cheng Zhang, Loren Mear, Wen Zhong, Andreas Digre, Borbala Katona, Evelina Sjöstedt, Lynn Butle, Jacob Odeberg, Philip Dusart. (2021). A single-cell type transcriptomics map of human tissues. Sci Adv., 7, (31).
16. Vuong Tran, Efthymia Papalexi, Sarah Schroeder, Grace Kim, Ajay Sapre, Joey Pangallo, Alex Sova, Peter Matulich, Lauren Kenyon, Zeynep Sayar. (2022) High sensitivity single cell RNA sequencing with split pool barcoding. Biorxiv. https://doi.org/10.1101/2022.08.27.505512
17. Binyamin D. Berkovits & Christine Mayr. (2015) Alternative 3′ UTRs act as scaffolds to regulate membrane protein localization. Nature., 522, 363-367.
18. Shih-Han Lee, Christine Mayr. (2019) Gain of Additional BIRC3 Protein Functions through 3′-UTR-Mediated Protein Complex Formation. Mol Cell. 74, (4), 701-712.e9.
19. Christine Mayr. (2017) Regulation by 3′-Untranslated Regions. Annu. Rev. Genet. 51, 171-194.
20. Jeffrey R Moffitt, Junjie Hao, Guiping Wang, Kok Hao Chen, Hazen P Babcock, Xiaowei Zhuang. (2016) High-throughput single-cell gene-expression profiling with multiplexed error-robust fluorescence in situ hybridization. Proc. Natl. Acad. Sci. U.S.A., 113, (39), 11046-11051.

It is understood that the examples and embodiments described herein are for illustrative purposes only and that various modifications or changes in light thereof will be suggested to persons skilled in the art and are to be included within the spirit and purview of this application and scope of the appended claims.

All publications, patents, and patent applications cited herein are hereby incorporated by reference with respect to the material for which they are expressly cited.

What is claimed is:

1. A method of in situ detection of RNA in cells, the method comprising,
providing fixed and permeabilized cells in a bulk solution;
in the bulk solution, diffusing single-stranded (ss) DNA probe pairs into the cells and annealing the ssDNA probe pairs to RNA in the cells, wherein the probe pairs comprise a 5′ 4 binding probe and a 3′ binding probe, wherein the 5′ binding probe and the 3′ binding probe anneal to adjacent sequences in a target RNA and wherein,
the 5′ binding probe comprises a 5′ universal sequence that does not anneal to the target RNA and a 3′ RNA annealing sequence and,
the 3′ binding probe comprises a 5′ phosphorylation, a 5′ RNA annealing sequence, and a 3′ adapter sequence that does not anneal to the target RNA;
washing unbound ssDNA probes from the cells;
ligating in the cells annealed probe pairs such that adjacent annealed 5′ binding probes and annealed 3′ binding probes are ligated to form one long probe;
performing a plurality of split-pooling rounds, wherein each round comprises:
(a) aliquoting the cells into a plurality of vessels,
(b) in the vessels, hybridizing a double-stranded (ds) barcoding oligonucleotide comprising (i) a first overhang sequence and (ii) a central double-stranded sequence having a barcode sequence and (iii) a second overhang sequence, wherein the first overhang anneals to a 3′ or 5′ end of the long probe and,
(c) in the vessels, ligating a strand of the ds barcoding oligonucleotide to the 3′ or 5′ end of the long probe to form barcoded ligated products, and
(d) combining the contents of the vessels to form a bulk solution comprising cells containing the ligated products,
wherein the plurality of split-pooling rounds forms cell-specific barcoded long probe polynucleotides; and
nucleotide sequencing the cell-specific barcoded long probe polynucleotides.

2. The method of claim 1, wherein the hybridizing comprises hybridizing a double-stranded (ds) barcoding oligonucleotide comprising (i) a 3′ first overhang sequence and (ii) a central double-stranded sequence having a barcode sequence and (iii) a second 3′ overhang sequence, wherein the 3′ first overhang anneals to a 3′ end of the long probe; and
the ligating comprises ligating a strand of the ds barcoding oligonucleotide to the 3′ end of the long probe to form barcoded ligated products.

3. The method of claim 1, wherein the hybridizing comprises hybridizing a double-stranded (ds) barcoding oligonucleotide comprising (i) a 5′ first overhang sequence and (ii) a central double-stranded sequence having a barcode sequence and (iii) a second 5′ overhang sequence, wherein the 5′ first overhang anneals to a 5′ end of the long probe; and
the ligating comprises ligating a strand of the ds barcoding oligonucleotide to the 5′ end of the long probe to form barcoded ligated products.

4. The method of claim 1, wherein a first round of split-pooling comprises:
aliquoting cells from the bulk solution into a plurality of vessels;
in the vessels annealing a first double-stranded (ds) barcoding oligonucleotide comprising (i) a 3′ overhang sequence that anneals to the 3′ adapter sequence on the long probe and (ii) a central double-stranded sequence having a first barcode sequence and (iii) a 3′ overhang sequence comprising a first linking sequence,
in the vessels, ligating the first ds barcoding oligonucleotide to the 3′ adapter sequence on the long probe to form a first partially barcoded long probe comprising the first barcode sequence and a 3′ end having the first linking sequence;
combining the contents of the vessels to form a second bulk solution.

5. The method of claim 4, further comprising a second round of split-pooling after the first round, the second round comprising,
aliquoting cells from the second bulk solution into a new plurality of vessels;
in the vessels annealing a second double-stranded (ds) barcoding oligonucleotide comprising (i) a 3′ overhang sequence that anneals to the first linking sequence and (ii) a central double-stranded sequence having a second barcode sequence and (iii) a 3′ overhang sequence comprising a second linking sequence;
in the vessels, ligating the second ds barcoding oligonucleotide to the first linking sequence on the long probe to form a second partially long probe comprising the first and second barcode sequence and a 3′ end having the second linking sequence; and
combining the contents of the vessels to form a third bulk solution.

6. The method of claim 4, further comprising a third round of split-pooling after the second round, the third round comprising,
aliquoting cells from the third bulk solution into a new plurality of vessels;
in the vessels annealing a third double-stranded (ds) barcoding oligonucleotide comprising (i) a 3′ overhang sequence that anneals to the second linking sequence and (ii) a central double-stranded sequence having a third barcode sequence and (iii) a 3′ overhang sequence comprising a third linking sequence;
in the vessels, ligating the third ds barcoding oligonucleotide to the second linking sequence on the long probe to form a third long probe comprising the first and second and third barcode sequence and a 3′ end having the third linking sequence; and
combining the contents of the vessels to form a fourth bulk solution.

7. The method of claim 1, further comprising, before the nucleotide sequencing, amplifying the cell-specific barcoded long probe polynucleotides with (i) a first primer that anneals to the 5′ universal sequence or a complement thereof and (ii) a second primer that anneals to a 3′ sequence of the cell-specific barcoded long probe polynucleotides or a complement thereof to form an amplicon.

8. The method of claim 1, wherein a first round of split-pooling comprises:
aliquoting cells from the bulk solution into a plurality of vessels;
in the vessels annealing a first double-stranded (ds) barcoding oligonucleotide comprising (i) a 5′ overhang sequence that anneals to the 5′ universal sequence on the long probe and (ii) a central double-stranded sequence having a first barcode sequence and (iii) a 5′ overhang sequence comprising a first linking sequence,
in the vessels, ligating the first ds barcoding oligonucleotide to the 5′ universal sequence on the long probe to form a first partially barcoded long probe comprising the first barcode sequence and a 5′ end having the first linking sequence;
combining the contents of the vessels to form a second bulk solution.

9. The method of claim 4, further comprising a second round of split-pooling after the first round, the second round comprising,
aliquoting cells from the second bulk solution into a new plurality of vessels;
in the vessels annealing a second double-stranded (ds) barcoding oligonucleotide comprising (i) a 5′ overhang sequence that anneals to the first linking sequence and (ii) a central double-stranded sequence having a second barcode sequence and (iii) a 5′ overhang sequence comprising a second linking sequence;
in the vessels, ligating the second ds barcoding oligonucleotide to the first linking sequence on the long probe to form a second partially long probe comprising the first and second barcode sequence and a 5′ end having the second linking sequence; and
combining the contents of the vessels to form a third bulk solution.

10. The method of claim 4, further comprising a third round of split-pooling after the second round, the third round comprising,
aliquoting cells from the third bulk solution into a new plurality of vessels;
in the vessels annealing a third double-stranded (ds) barcoding oligonucleotide comprising (i) a 5′ overhang sequence that anneals to the second linking sequence and (ii) a central double-stranded sequence having a third barcode sequence and (iii) a 5′ overhang sequence comprising a third linking sequence;
in the vessels, ligating the third ds barcoding oligonucleotide to the second linking sequence on the long probe to form a third long probe comprising the first and second and third barcode sequence and a 5′ end having the third linking sequence; and
combining the contents of the vessels to form a fourth bulk solution.

11. The method of claim 1, further comprising, before the nucleotide sequencing, amplifying the cell-specific barcoded long probe polynucleotides with (i) a first primer that anneals to the 3′ adapter sequence or a complement thereof and (ii) a second primer that anneals to a 5′ sequence of the cell-specific barcoded long probe polynucleotides or a complement thereof to form an amplicon.

12. The method of claim 7, wherein the amplifying occurs in a plurality of vessels.

13. The method of claim 12, wherein the second primers comprise a further vessel-specific barcoding sequence.

14. The method of claim 13, wherein the first primer and the second primers comprise 5′ sequences that introduce sequencing adapter sequences to the amplicon.

15. The method of claim 1, wherein at least 2 or more different ss DNA probe pairs are targeted to different sequences on the same target RNA.

16. The method of claim 1, wherein different ss DNA probe pairs are targeted to different target RNAs.

17. The method of claim 1, wherein a PBCV-1 DNA ligase catalyzes the ligating of the annealed probe pairs in the cells.

18. The method of claim 1, wherein the first and second nucleotide of the 5′ RNA annealing sequence is A or T.

19. A reaction mixture comprising fixed and permeabilized cells and single-stranded (ss) DNA probe pairs diffused into the cells, wherein at least some of the ss DNA probe pairs anneal to RNA in the cells, wherein the probe pairs comprise a 5′ binding probe and a 3′ binding probe, wherein the 5′ binding probe and the 3′ binding probe anneal to adjacent sequences in a target RNA and wherein,
the 5′ binding probe comprises a 5′ universal sequence that does not anneal to the target RNA and a 3′ RNA annealing sequence and,
the 3′ binding probe comprises a 5′ phosphorylation, a 5′ RNA annealing sequence, and a 3′ adapter sequence that does not anneal to the target RNA.

20. A kit comprising,
at least 100 different single-stranded (ss) DNA probe pairs that anneal to different RNA from a cell, wherein the probe pairs comprise a 5′ binding probe and a 3′ binding probe, wherein the 5′ binding probe and the 3′ binding probe anneal to adjacent sequences in a target RNA and wherein, the 5′ binding probe comprises a 5′ universal sequence that does not anneal to the target RNA and a 3′ RNA annealing sequence and, the 3′ binding probe comprises a 5′ phosphorylation, a 5′ RNA annealing sequence, and a 3′ adapter sequence that does not anneal to the target RNA; and
a plurality of double-stranded (ds) barcoding oligonucleotides comprising (i) a first overhang sequence and (ii) a central double-stranded sequence having a barcode sequence and (iii) a second overhang sequence.
